SEQUENCE LISTING (101 sequences)

SEQ ID NO: 1 - DNA, 100 bp, Homo sapiens
gctgggatttacagctaaggttttatggcttctggaaaccctgagccagatgagtgagattcccagagcaggagaccaagactagctggtctctcacacc

SEQ ID NO: 2 - DNA, 100 bp, homo sapiens
gctgggatttacagctaaggttttatggcttctggaaaccctgagccagaggagtgagattcccagagcaggagaccaagactagctggtctctcacacc

SEQ ID NO: 3 - protein, 193 aa, homo sapiens [sequence omitted]

SEQ ID NO: 4 - DNA, 3398 bp, homo sapiens
agcttctgggcttccagacccagctactttgcggaactcagcaacccaggcatctctgagtctccgcccaagaccgggatgccccccaggaggtgtccgggagcccagcctttcccagatagcagctccgccagtcccaagggtgcgcaaccggctgcactcccctcccgcgacccagggcccgggagcagcccccatgacccacacgcacgtctgcagcagccccgtcagccccggagcctcaacccaggcgtcctgcccctgctctgaccccgggtggcccctacccctggcgacccctcacgcacacagcctctcccccacccccacccgcgcacgcacacatgcagataacagccccgacccccggccagagccgcagagtccctgggccaccccggccgctcgctgcgctgcgccgcaccgcgctgtcctcccggagccggaccggggccaccgcgcccgctctgctccgacaccgcgccccctggacagccgccctctcctccaggcccgtggggctggccctgcaccgccgagcttcccgggatgagggcccccggtgtggtcacccggcgccccaggtcgctgagggaccccggccaggcgcggagatgggggtgcacggtgagtactcgcgggctgggcgctcccgcccgcccgggtccctgtttgagcggggatttagcgccccggctattggccaggaggtggctgggttcaaggaccggcgacttgtcaaggaccccggaagggggaggggggtggggcagcctccacgtgccagcggggacttgggggagtccttggggatggcaaaaacctgacctgtgaaggggacacagtttgggggttgaggggaagaaggtttggggggttctgctgtgccagtggagaggaagctgataagctgataacctgggcgctggagccaccacttatctgccagaggggaagcctctgtcacaccaggattgaagtttggccggagaagtggatgctggtagcctgggggtggggtgtgcacacggcagcaggattgaatgaaggccagggaggcagcacctgagtgcttgcatggttggggacaggaaggacgagctggggcagagacgtggggatgaaggaagctgtccttccacagccacccttctccctccccgcctgactctcagcctggctatctgttctagaatgtcctgcctggctgtggcttctcctgtccctgctgtcgctccctctgggcctcccagtcctgggcgccccaccacgcctcatctgtgacagccgagtcctgcagaggtacctcttggaggccaaggaggccgagaatatcacggtgagaccccttccccagcacattccacagaactcacgctcagggcttcagggaactcctcccagatccaggaacctggcacttggtttggggtggagttgggaagctagacactgcccccctacataagaataagtctggtggccccaaaccatacctggaaactaggcaaggagcaaagccagcagatcctacgcctgtggccagggccagagccttcagggacccttgactccccgggctgtgtgcatttcagacgggctgtgctgaacactgcagcttgaatgagaatatcactgtcccagacaccaaagttaatttctatgcctggaagaggatggaggtgagttcctttttttttttttttcctttcttttggagaatctcatttgcgagcctgattttggatgaaagggagaatgatcgagggaaaggtaaaatggagcagcagagatgaggctgcctgggcgcagaggctcacgtctataatcccaggctgagatggccgagatgggagaattgcttgagccctggagtttcagaccaacctaggcagcatagtgagatcccccatctctacaaacatttaaaaaaattagtcaggtgaagtggtgcatggtggtagtcccagatatttggaaggctgaggcgggaggatcgcttgagcccaggaatttgaggctgcagtgagctgtgatcacaccactgcactccagcctcagtgacagagtgaggccctgtctcaaaaaagaaaagaaaaaagaaaaataatgagggctgtatggaatacgttcattattcattcactcactcactcactcattcattcattcattcattcaacaagtcttattgcataccttctgtttgctcagcttggtgcttggggctgctgaggggcaggagggagagggtgacatccctcagctgactcccagagtccactccctgtaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctgtaagaaggggagaagggtcttgctaaggagtagaggaactgtccgtattccttccctttctgtggcactgcagcgacctcctgttttctccttggcagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccaccaatgacatctcaggggccagaggaactgtccagagagcaactctgagatctaaggatgtcacagggccaacttgagggcccagagcaggaagcattcagagagcagctttaaactcagggacagacccatgctgggaagacgcctgagctcactcggcaccctgcaaaattgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaacttttgtgtattcttcaacctcattgacaagaactgaaaccaccaatatgactcttggcttttctgttttctgggaacctccaaatcccctggctctgtcccactcctggcagca

SEQ ID NO: 5 - DNA, 20 bp, Artificial Sequence
  completely synthesized
ccaccctgctgcttttactc

SEQ ID NO: 6 - DNA, 20 bp, Artificial Sequence
  completely synthesized
ctcagtctgggaccttctgc

SEQ ID NO: 7 - DNA, 20 bp, Artificial Sequence
  completely synthesized
gtgaaggtcggtgtgaacgg

SEQ ID NO: 8 - DNA, 20 bp, Artificial Sequence
  completely synthesized
gccgttgaatttgccgtgag

SEQ ID NO: 9 - DNA, 29 bp, Artificial Sequence
  completely synthesized
cggggtaccaactcctgggctcaaaggat

SEQ ID NO: 10 - DNA, 34 bp, Artificial Sequence
  completely synthesized
ccgctcgagtccgcgcctggccggggtccctcag

SEQ ID NO: 11 - DNA, 34 bp, Artificial Sequence
  completely synthesized
cggggtacccctgcacaatggactgtgtgctctg

SEQ ID NO: 12 - DNA, 34 bp, Artificial Sequence; identical to SEQ ID NO: 10
  completely synthesized

SEQ ID NO: 13 - DNA, 20 bp, Artificial Sequence
  completely synthesized
aagttggaaggaccacatgc

SEQ ID NO: 14 - DNA, 20 bp, Artificial Sequence
  completely synthesized
tagctgagcgagcacacact

SEQ ID NO: 15 - DNA, 40 bp, Artificial Sequence
  completely synthesized
ggattactttgcagtcccagcaacatggtgggctggactc

SEQ ID NO: 16 - DNA, 20 bp, Artificial Sequence
  completely synthesized
gatgagcaggatgcatgaga

SEQ ID NO: 17 - DNA, 20 bp, Artificial Sequence
  completely synthesized
cttgatgtctgtgggggact

SEQ ID NO: 18 - DNA, 28 bp, Artificial Sequence
  completely synthesized
gtaaatagttgctatactgtattgtttt

SEQ ID NO: 19 - DNA, 20 bp, Artificial Sequence
  completely synthesized
gtccattgtgcaggacacac

SEQ ID NO: 20 - DNA, 20 bp, Artificial Sequence
  completely synthesized
aactcctgggctcaaaggat

SEQ ID NO: 21 - DNA, 28 bp, Artificial Sequence
  completely synthesized
ttatggcttctggaaaccctgagccaga

SEQ ID NO: 22 - DNA, 20 bp, Artificial Sequence
  completely synthesized
gactcagtggcttggaggag

SEQ ID NO: 23 - DNA, 20 bp, Artificial Sequence
  completely synthesized
gccagatttggaagcatcat

SEQ ID NO: 24 - DNA, 34 bp, Artificial Sequence
  completely synthesized
ttaaccacttcctagctctgttcacatcaaatcg

SEQ ID NO: 25 - DNA, 20 bp, Artificial Sequence
  completely synthesized
ggctgtgcaatttaccacct

SEQ ID NO: 26 - DNA, 19 bp, Artificial Sequence
  completely synthesized
gtcacctgttccctgctgt

SEQ ID NO: 27 - DNA, 46 bp, Artificial Sequence
  completely synthesized
gacacaggctacttgttccgaagttttaagctgtagatgttcccaa

SEQ ID NO: 28 - DNA, 20 bp, Artificial Sequence
  completely synthesized
aggagaatgctgcctctgaa

SEQ ID NO: 29 - DNA, 20 bp, Artificial Sequence
  completely synthesized
tctcgatctcctgacctcgt

SEQ ID NO: 30 - DNA, 34 bp, Artificial Sequence
  completely synthesized
aatatctatctagtggaaacacaaaatgcttata

SEQ ID NO: 31 - DNA, 20 bp, Artificial Sequence
  completely synthesized
aacctccccacagaaaggat

SEQ ID NO: 32 - DNA, 20 bp, Artificial Sequence
  completely synthesized
cccctggattgtcactcatt

SEQ ID NO: 33 - DNA, 52 bp, Artificial Sequence
  completely synthesized
atttcttctgaatgaagacctttggatttctaaaagcaccaaaacaaggctt

SEQ ID NO: 34 - DNA, 20 bp, Artificial Sequence
  completely synthesized
tcctgaatccagaccctcac

SEQ ID NO: 35 - DNA, 20 bp, Artificial Sequence
  completely synthesized
gtccctctgggagtttcctc

SEQ ID NO: 36 - DNA, 40 bp, Artificial Sequence
  completely synthesized
ttttgattctttccaggctcatgaacttgaaagcatttac

SEQ ID NO: 37 - DNA, 20 bp, Artificial Sequence
  completely synthesized
gccaccgaagagtggaaata

SEQ ID NO: 38 - DNA, 20 bp, Artificial Sequence
  completely synthesized
ctacttgggaggctgaggtg

SEQ ID NO: 39 - DNA, 28 bp, Artificial Sequence
  completely synthesized
aaattgatgaaaatggtaaactgtggcc

SEQ ID NO: 40 - DNA, 20 bp, Artificial Sequence
  completely synthesized
gcggatgaaaaggaaactga

SEQ ID NO: 41 - DNA, 20 bp, Artificial Sequence
  completely synthesized
ttttggtctaccctgccaag

SEQ ID NO: 42 - DNA, 20 bp, Artificial Sequence
  completely synthesized
atgcagcagcctctaagtca

SEQ ID NO: 43 - DNA, 20 bp, Artificial Sequence
  completely synthesized
ccctcccccatgaaaactat

SEQ ID NO: 44 - DNA, 20 bp, Artificial Sequence
  completely synthesized
ttcacagaagtgagcggttg

SEQ ID NO: 45 - DNA, 40 bp, Artificial Sequence
  completely synthesized
gtatgatttctcctgccaatatcataagctgagaatattt

SEQ ID NO: 46 - DNA, 20 bp, Artificial Sequence
  completely synthesized
aggcagaagtttccctggtt

SEQ ID NO: 47 - DNA, 20 bp, Artificial Sequence
  completely synthesized
aatccatgtgggagagttgc

SEQ ID NO: 48 - DNA, 28 bp, Artificial Sequence
  completely synthesized
ttagcttctttcttcctcaaggactttc

SEQ ID NO: 49 - DNA, 21 bp, Artificial Sequence
  completely synthesized
ccactacggcctcaatgagag

SEQ ID NO: 50 - DNA, 20 bp, Artificial Sequence
  completely synthesized
tgccaagtattgcatcgaga

SEQ ID NO: 51 - DNA, 33 bp, Artificial Sequence
  completely synthesized
taaatagagaattacttccctaggaccaccaga

SEQ ID NO: 52 - DNA, 20 bp, Artificial Sequence
  completely synthesized
gtagaagggcacaagcaagc

SEQ ID NO: 53 - DNA, 19 bp, Artificial Sequence
  completely synthesized
aggaaacctgctttgctga

SEQ ID NO: 54 - DNA, 41 bp, Artificial Sequence
  completely synthesized
acatagcaattcatcaaacaacagcgtgcttccaagtcacc

SEQ ID NO: 55 - DNA, 20 bp, Artificial Sequence
  completely synthesized
ctgagcgctaaaccagaacc

SEQ ID NO: 56 - DNA, 21 bp, Artificial Sequence
  completely synthesized
gcaccactgcacttccagtta

SEQ ID NO: 57 - DNA, 34 bp, Artificial Sequence
  completely synthesized
ctcaaagacgggatgcttgtcgtcgagtctcata

SEQ ID NO: 58 - DNA, 20 bp, Artificial Sequence
  completely synthesized
tagattctggaattcctgtg

SEQ ID NO: 59 - DNA, 20 bp, Artificial Sequence
  completely synthesized
ggtaatttccaagatacaac

SEQ ID NO: 60 - DNA, 52 bp, Artificial Sequence
  completely synthesized
ataggcttgagtgggagcttttgttctgtgtcttaaaattattgatattgtc

SEQ ID NO: 61 - DNA, 20 bp, Artificial Sequence
  completely synthesized
gcctggattctggtgttgat

SEQ ID NO: 62 - DNA, 20 bp, Artificial Sequence
  completely synthesized
acgctgggtgtagggactta

SEQ ID NO: 63 - DNA, 57 bp, Artificial Sequence
  completely synthesized
tcgttgtatgaattttagcattacagataccacacctattagatatgggcaggacta

SEQ ID NO: 64 - DNA, 20 bp, Artificial Sequence
  completely synthesized
tactcagcaaaccccaaagg

SEQ ID NO: 65 - DNA, 20 bp, Artificial Sequence
  completely synthesized
caatcctcttctccccaaca

SEQ ID NO: 66 - DNA, 45 bp, Artificial Sequence
  completely synthesized
cagagagggagaggcgggagatgtcagagacggagacgaggcaac

SEQ ID NO: 67 - DNA, 20 bp, Artificial Sequence
  completely synthesized
tcctgccaactcacctctct

SEQ ID NO: 68 - DNA, 20 bp, Artificial Sequence
  completely synthesized
agggagacccagatggagat

SEQ ID NO: 69 - DNA, 46 bp, Artificial Sequence
  completely synthesized
cgcacgcagcagttcttctccgtggagctgcaggcaggagagacgc

SEQ ID NO: 70 - DNA, 20 bp, Artificial Sequence
  completely synthesized
acccaaatgatcctccttcc

SEQ ID NO: 71 - DNA, 20 bp, Artificial Sequence
  completely synthesized
acaccaggcactcaaattcc

SEQ ID NO: 72 - DNA, 34 bp, Artificial Sequence
  completely synthesized
ctttggaagttactccagaatgtataccttactt

SEQ ID NO: 73 - DNA, 20 bp, Artificial Sequence
  completely synthesized
acctggctttctggatgcta

SEQ ID NO: 74 - DNA, 20 bp, Artificial Sequence
  completely synthesized
tcatcttgagggatccaagg

SEQ ID NO: 75 - DNA, 40 bp, Artificial Sequence
  completely synthesized
gatgtgagccactgtgcccgacccatatcctgttccaata

SEQ ID NO: 76 - DNA, 19 bp, Artificial Sequence
  completely synthesized
gctgacctcagagcattcc

SEQ ID NO: 77 - DNA, 20 bp, Artificial Sequence
  completely synthesized
tcacataattgttgggagcg

SEQ ID NO: 78 - DNA, 53 bp, Artificial Sequence
  completely synthesized
ccctgcaactgctacaattaacaggatatgggagagaggaggcacatcatgtg

SEQ ID NO: 79 - DNA, 20 bp, Artificial Sequence
  completely synthesized
taatccaggatgagccaagg

SEQ ID NO: 80 - DNA, 20 bp, Artificial Sequence
  completely synthesized
ctcccaaagtgctgggatta

SEQ ID NO: 81 - DNA, 34 bp, Artificial Sequence
  completely synthesized
gagggtctcagttctgaggcaataggaagccgtg

SEQ ID NO: 82 - DNA, 20 bp, Artificial Sequence
  completely synthesized
cctgttttcgcacctaccat

SEQ ID NO: 83 - DNA, 20 bp, Artificial Sequence
  completely synthesized
tagcgaggatgtgcattgag

SEQ ID NO: 84 - DNA, 46 bp, Artificial Sequence
  completely synthesized
gggcagggttggcagctgccttactgcggtgaggccttgaatggag

SEQ ID NO: 85 - DNA, 20 bp, Artificial Sequence
  completely synthesized
tcccaaagtgctgggattac

SEQ ID NO: 86 - DNA, 20 bp, Artificial Sequence
  completely synthesized
gtcaggctggtctcgaactc

SEQ ID NO: 87 - DNA, 28 bp, Artificial Sequence
  completely synthesized
agacactggaatctaagctgaaggctaa

SEQ ID NO: 88 - DNA, 20 bp, Artificial Sequence
  completely synthesized
gggtgtcagagcaagactcc

SEQ ID NO: 89 - DNA, 20 bp, Artificial Sequence
  completely synthesized
agtggggacacacagaggac

SEQ ID NO: 90 - DNA, 46 bp, Artificial Sequence
  completely synthesized
gttgttgttgtagttccctagaggagggagtcattaaaacaggcaa

SEQ ID NO: 91 - DNA, 20 bp, Artificial Sequence
  completely synthesized
gcctttctgtgtagggtgga

SEQ ID NO: 92 - DNA, 20 bp, Artificial Sequence
  completely synthesized
gctgggcatcattagttcgt

SEQ ID NO: 93 - DNA, 46 bp, Artificial Sequence; identical to SEQ ID NO: 90
  completely synthesized

SEQ ID NO: 94 - DNA, 20 bp, Artificial Sequence
  completely synthesized
aggagttcaaggctgcagaa

SEQ ID NO: 95 - DNA, 20 bp, Artificial Sequence
  completely synthesized
tctctccttcccaggactca

SEQ ID NO: 96 - DNA, 58 bp, Artificial Sequence
  completely synthesized
ctctctggtcacggccagtagtacgaccaccacaggcaccagcagtcccagtaggacg

SEQ ID NO: 97 - DNA, 21 bp, Homo sapiens
ctgagccagaggagtgagatt

SEQ ID NO: 98 - DNA, 17 bp, Homo sapiens
tgagccagatgagtgag

SEQ ID NO: 99 - DNA, 10 bp, Homo sapiens
gatgagtgag

SEQ ID NO: 100 - protein, 507 aa, Homo sapiens
MDHLGASLWPGVGSLCLLLAGAAWAPPPNLPDPKFESKAAILAARGPEELICFTERLEDLVCFWEEAASAGVGPGNYSFSYQLEDEPWKLCRLHGAPTARGAVRFWCSLPTADTSSFVPLELRVTAASGAPRYHRVIHINEVVLIDAPVGIVARLADESGHVVLRWLPPPETPMTSHIRYEVDVSAGNGAGSVQRVEILEGRTECVISNLRGRTRYTFAVRARMAEPSFGGFWSAWSEPVSLLTPSDLDPLILTLSLIIVVILVLLTVLAILSHRRALKQKIWPGIPSPESEFEGLFTTHKGNFGLWLYQNDGCLWWSPCTPFTEDPPASLEVLSERCWGTMQAVEPGTDDEGPLLEPVGSEHAQDTYLVLDKWLLPRNPPSEDIPGPGGSVDIVAMDEGSEASSCSSALASKPSPEGASAASFEYTILDPSSGLLRPWTICPEIPPTPPHLKYLYLVVSDSGISTDYSSGDSQGAQGGLSDGPYSNPYENSLIPAAEPLPPSYVAC

SEQ ID NO: 101 - DNA, 8647 bp, Homo sapiens
ggatccacccacctcggcctcccaaagtgctgggattacaggcatgagcactgtgcatggactatttatttatttttttgaaacagagtttcaatcttgttgcacagcctggagtgcaatggtgtgatctcagctcactgcaacctctgccttctggtttcaagcaattctcctgcctcagcctcctgagtagctgggattacaggcacccaccaccacgctcgaatatatatatatattttttgagacggagtccgctctgtcaccaggctggagtgcagtggccaaatatcggctcactgaaacctccggctcctgggttcaagcgattctcctgcagcctcccaagtagctgggattacaggcatgcagcaccacgcccatctaatttttgtatttttggtagagatggggttttaccatgttggccaggatggtcttgatctcttgacctcgtgatctgcccacctcggcctcccaaagtgctgggattacaggcgtgacgaccgcgcccggcctacgcctggctaatttttgtatttttagtagagacgtggtttcgccatgttgcccaggctggtctcgaactcctgacctcatgatccgcctgtctcggcctcccaaagtgttgggattacaagtatgagccaccgcgccactagccaattttttttattttttgagatgcagtctcactctgttgcccaggctggagttgcagtggcatgatcttggctcactgcaatcttcatctcccagactgaagcagttctcatgcctcagcctcctgagtagctgggattacagcacacgccaccacacctggctaatttttgtatttttagtagagatgggatttcaccatgttggccaggctggtctcaaactcctgacctcaagtgatttgcccacgtcggcctcccaaagtgctgggattataggcgtgagccaccgcccagcccaagagaataaaaatgtgggtggtaaaaatttttttcccaaaaattcgtaaatgaaaatctcacatattatgcatactgcccaggagcatggcctagcactgtgcaaacactcaactgctggtcgttgcaaggattattattggccggcttcagtggcttgctggtattcccagcacattgggagatggaggctggaggattgcttaagtccgggatttcaagaccagcctggacaacatagtgggatcccatctctacaaagaattttaaaaattagccaggtgcagtgggaagattgcttcagtccagaggctgcagtgagctatgattgtgccactgcactccagcctgggtgacagagcaacaccctgagacagagagagagagggggaaggagggaaggaaggaaggaaggaaggaaggaaggaaggaaggaaggaaggaaggaaggaaggaaaggagagagagagagagagagagagagagagagagagaaaataatttttatttatttccaggctgggaagagatgctgatttctgcgataaaatcagtaggtacattttttggaatgttcgctatgtgccaggctagattttacagatgagaagtctgaagctcaggtaaggtaagtcacctgtccagggccacaaagaaaaaaaaaacgtgtgtctgaagccagaacgggagctgttgcgcccaactccctcccctgcccccaagcggcctctgggctcgggaagggcccctgcctcctcccgccaggcacttatctctacccaggctgagtgctggccccgcccctcggggatctgccacttagaggcgcctggtcgggaagggcctggtcagctgcgtccggcggaggcagctgctgacccagctgtggactgtgccgggggtgggggacgcaggggcaggagccctgggctccccgtggcgggggctgtatcatggaccacctcggggcgtccctctggccccaggtcggctccctttgtctcctgctcgctggggccgcctgggcgcccccgcctaacctcccggaccccaagttcgagagcaaaggtaaggatgagctgcgtgtggacccctacgctggagcctgcaggaccatgctggggcctgaactcccagcctaggtcctgggggccatgctgtttctggacttcctgaccgggtcctgggggccaagctggcatctgaacccttagactgggtcctggatgggtggggggcggggtggggtatgttaggatccaagactcctgatcccgtcccgggcaagagctagagtgggcttaacattcccgttttaccttttcagggagtctgggacatgctaaatcctaagggggctgacttggtgctaaggtccctggggggtggggaccaagccgatccctaggggagggagggtaaagcccgggtccgagttagagggccaagccacaggctactgtaaacacggtttgtgtgagggcgccagatcacttgcccggcccggtggagggagggaggcggggggcacggttgccgctatcggttggcggggagcctgccggggccgatagggggcccgcctctccgcacacacccccagccgcgcgcgtgtcctaggctggggcggggctggcagtcccgagctcgaggtcttgaacgccgcgcccagctcagctggccgctgggtgggcaggtgtgcgccagtggtgcacggcgggggacagtaaggcgagaaacttgcccctgggaattaggggggcaccacctctgcggacccctccaagggacccgcttgggaagatggcagggcggggcttttttcttatcgggtccgcccaggctgcgggagggaagaggagggggctgtctcccgaggatagagctcagacccccatgcccttcctttgtcgcccctccccagcggccttgctggcggcccgggggcccgaagagcttctgtgcttcaccgagcggttggaggacttggtgtgtttctgggaggaagcggcgagcgctggggtgggcccgggcaactacagcttctcctaccagctcgagtgagtccgatccggcgggtgcctccaagggcggagggagggggtggggcagagctccctggaggtcgtagcctcgtatgtcccctgctgtttgaggcccgacggcgcctccagtcgtggtcactggagggaaacctgcgggtccagggctggcacgcctctatgggccggggcgcgaacactcccgcgatcaccgctggaacgcgaccccaaacatcaggctgggataacaacgcctccaaatcgagggtaaggcgttactacgtcggggctgggacgccttctcgaggtagtatccaaaaggaggccagcagtgctcatgcctgtaatcccaactctttggaaggtccagcggaagaaccgcttgagcccaggtgttcaagaccagcctgggcaacacagcgagatccccgtctcttaaaaaaaaattagactgggcgcggctgcacgcctgtaatcccagcactttgggaggctgaggcgggcggatcacctgaggtcgggagtttgagagccagcctggccaacatggagaaactctatctctactaaaaatacaaaattagccgggcgtggtggcgcatgcctgtgatcccagctactcgggaggctgaggcaggagaatcgcttgaacccgggaggcggaggttgcggtgagccgaggtagcgccattgcactccagcctgggcaacaagagcgaaactccgtctcaaaaaaaaaaaaaataaaagccaggcgtggcgcgtgcctgtggtctcaactacttgggaagctgaggtgggaggatcccttaagccccagaatttgaggctccagtgagccataatcgcgccactgcactccagcctgggcgacgaaggaacaccttgtcacacacacacacaaggctagaccttgtgtcacacatacacactgccccccacaggccgggcaatgccaactccccggtcccccctcccaacctgctcccttccctgggcgcatagggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtaggtaagtgctctgggaatggaggagtggtcggaggagagggtctcagtcctcgcccacctgaccaacccccatgcctgcagtgctcctagacgcccccgtggggctggtggcccggttggctgacgagagcggccacgtagtgttgcgctggctcccgccgcctgagacacccatgacgtctcacatccgctacgaggtggacgtctcggccggcaacggcgcagggagcgtacagagggtgaggccactccctacggcccagcccccaaagctccactgactacggcccagccacgcctctcgaggtcgcgcccggtgccgctttcagggccggtccgtaacatcccacatcccattaccctggtgctgaagaccgttccacgcccacagacacagccccctttcctaatgtcctcgcaagcctgttgaaccccaacttcttctccctccgtcccgtaaccctagacccctttagcgcccgggtccctctacgagtgctagcccagatattaaattgcccgggtcccgccctttcgtaccagagactctctctctgattggccctgagctttcttgggctcctccccctactcttattggtcccattgcaattctagggcaccgttttcctttcccctgattggctcagttccaccagggcccgcccccacgtcatctatttttgtctgctacgcgtccctcgccctgattccgcccccaggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcggtgaggccccaggcgggggtgtaggaggagccagggcgaatcacggggcaagcccaccgccctgacctcctccccgcctcttagacctggaccccctcatcctgacgctctccctcatcctcgtggtcatcctggtgctgctgaccgtgctcgcgctgctctcccaccgccggtgagctccccatttgggcgctgggcccagactcctccccgccaacggtcctctttcactatggaaacctaggctcagagagagacacgcacttgcccaaggtcacgcagtaaggattcacatcagtggcagggctgggatgcatgccagactagacccagactcttcgttaacattttctgctcttggggactttcacctgattttccttctacatcaggggctgccatttcttgggtccctttgttacttcctttccccagtgtcatcacctttgtaaaatcaactagatggatttagtgaaagaatttaagaccctgaatgcctccgcacccctgcggtcaagcttctcagacactatgatcagactagccgttctgaggtatttgtaattccaagcacacactaggtggtttcacacccccaagcttttgcccatgctgttccctctgcctggaatgcccttcctgccttgtctgctaagcaatcttctagtcgtctttcatggccctgttcatttacttggttggaaaatacaaacagagtgccaaacatgtgccaggcactggagagagaatggagaacaagctagaccctgaccacaagtccctgaccttgtggatctcaagtcaacaaacaagggacccaagaaatatttgatgacaaattgtaatgagtgatatcacagaaacaaacagaatgtggtgacatgacaggatggtcagggaaggctccaggaggaggtgacatcagagtggaaacctgaagattggaaggaagcagccgcttgaaaagtggggagaagaaacagcaagtgcaaaggccctgaggtgggaatgagattggaacgttcagccagcttcaagaattgccacatgcatggcctggcatggtggctcacgcctgtaatcccagcactttgggatgccgaggcaggcacatcacctgaggttgggagttcgcgaccagcctgaccaacatggagaaaccccacctctactaaaaatacaaaactagccaagcgtggtggcacatgcctgtaatccccgctactcgggaggctgaggcaggagaatcacttgaacctgggaggtggaggttgcgggtgagccgagatcgtgccatcgcattccagcctgggcaataagagtgaaactccgtctcaaaaaaaaaaaaaaaattgccacatggctagagtggtatgtaagggggtgtggcagatattgagatgagggaggtgacaggggtcatataacgcagggccttctgcagggtggtggggaggagtttggaattttttttttttttgagacagagtcactcttctcgcccaagctgtagtgcagtgcagcagtcttggctcactgcaactctgcctcccaggttcaagtgattctcctgcctcaaccgcctgagtagctgagattacaggcgtgcatgcccggctaattttgtagttttagtagagacgggcttccaccatgttggccaggctggtctcaaactcctgacctcaggtgatctgctcacatcagcctctcaaagtgctgggattataggcatgagccaccgtgcctggcttggattttatcctaaatgcctctctcattaccccagaaggtaacataatatttatctatgaagtgacatcatggacctcctggaaaaatctgggccagggttttgggttttttaatttattttattttattttttttagagatgggggtctcactatgtttcctaggctggtcttgaactcctgggttcaaatgatcctcccacctcagcctcccaaagtactgggattatagtgctggtgtaaaccactgcacctggccatggccaggattaaagggagaatgaccaaggtatattgaactcctatgcacccttcaataccctgttccatttacccttttgtagggccttgctgatgcttcagccaaaacccctgtcccctggccctgatgtactcctctgcctccattgtgatcacagggaccaagtgtatctgtgcctctatgactgggagtggagggggaattggtgagtattcaatgagtcatatctatgtaactatttatattggcttcaacagggctctgaagcagaagatctggcctggcatcccgagcccagagagcgagtttgaaggcctcttcaccacccacaagggtaacttccaggtaggtggcctggttgtcccctcagtgcctgggcttccctgcttcttgcagccaaactgcaggcctctctgagcaggttggtgctatttcttcagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtacctttttgtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttttgtatctatatatatatatacacatatgtatgtaagtttttctaccatgatttctacaaacaccctttaagtcccatcttcccctgggcataggccatagggatagaagttaaagttcttgagcttattcagaagctggatctgcaatctgaatgctactcataacataacaaaatagtatgttaaacagctcttaaatcttactggcttaccacattaaatgatttctctctcctaactcagctcaaatgggcagccatccatggatgagtcagaggttcagactcttccagtctgtagctctaccttctcttagggtacttagatggatcc

(19) United States
(12) Patent Application Publication
(10) Pub. No.: US 2014/0120079 A1
(43) Pub. Date: May 1, 2014

(54) Compositions and Methods for Diagnosing and Treating Diabetic Micro Vascular Complications

(71) Applicant: University of Utah Research Foundation, Salt Lake City, UT (US)

(72) Inventor: Kang Zhang, Salt Lake City, UT (US)

(73) Assignee: University of Utah Research Foundation, Salt Lake City, UT (US)

(21) Appl. No.: 13/628,012

(22) Filed: Sep. 26, 2012

Related U.S. Application Data
(63) Continuation of application No. 12/530,354, filed on May 20, 2010, now abandoned.
(60) Provisional application No. 60/894,397, filed on Mar. 12, 2007.

(51) Int. Cl. C12Q 1/68; A61K 39/395; A61K 38/17; A61K 31/713

(52) U.S. Cl. CPC C12Q 1/6883; A61K 31/713; A61K 39/3955; A61K 38/17
USPC 424/130.1; 514/44 A; 514/1.1; 435/6.11

(57) ABSTRACT

Disclosed herein are compositions and methods for the identification of a subject at risk for developing microvascular complications associated with diabetes such as diabetic nephropathy and diabetic retinopathy. Also disclosed is a therapeutic target for the prevention and treatment of microvascular complications associated with diabetes.

### STATEMENT REGARDING FEDERALLY SPONSORED RESEARCH

This disclosure was made with government support under Grants R01EY14428, R01EY14448, P30EY014800, and GCRC M01-RR00064 awarded by the National Institutes of Health. As such, the government may have certain rights in the embodiments disclosed herein.

### BACKGROUND

The prevalence of diabetes is increasing steadily. Current prevalence in the US and worldwide (18 and 171 million persons respectively) are expected to increase to 30 and 366 million persons, respectively, by the year 2030. Despite the ability to treat diabetes with insulin therapy and oral hypoglycemic agents, diabetic complications remain a major worldwide cause of morbidity and mortality. Microvascular complications including retinopathy and nephropathy account for the most prevalent and severe morbidity associated with diabetes and may be involved in mediating the increased risk of cardio- and cerebrovascular disease as well. The sight-threatening form of diabetic retinopathy, termed proliferative diabetic retinopathy (PDR), is the most common cause of new-onset legal blindness in working-aged adults in the United States, accounting for 10% of new onset blindness overall. Diabetes is also the leading cause of renal insufficiency and end-stage renal disease (ESRD) in the U.S. and the Western world. Although diabetic microvascular complications are clearly associated with the degree of hyperglycemia, not all diabetic individuals with poor glycemic control develop renal or advanced retinal complications. Conversely, some diabetic patients develop severe complications despite well-controlled blood glucose concentrations (Reichard, P. 1995).

There is a high concordance in development of PDR and ESRD in diabetic patients. Although genetic factors appear to be important in the susceptibility (or resistance) to these complications, the causal gene(s) remain largely unknown. A growing number of studies have performed genome-wide scans to look for diabetic nephropathy susceptibility loci. These have included family-based linkage studies, concordant sib-pair, and discordant sib-pair studies. In addition, there have been a large number of case-control association studies examining the possible role of various candidate gene polymorphisms on diabetic nephropathy. Some loci have been identified in more than one analysis: for example, a discordant sib-pair study, a family-based analysis, and an association study have all suggested the existence of a diabetic nephropathy locus on chromosome 3q (Moczulski, D. K. 1998; Chistiakov et al., D. A. 2004; Bowden et al., D. W. 2004). Other loci for susceptibility to renal disease in the presence of diabetes have been suggested by additional studies (Rich, S. S. 2006).

PDR occurs in 30-50% of diabetic patients (Girach, A. 2006). PDR is characterized by neovascularization and fibrous proliferation which may result in traction retinal detachment, macular edema, and vitreous hemorrhage. The onset of PDR is thought to primarily arise after progressive retinal ischemia results in increased expression of the hypoxia-inducible vascular endothelial growth factor (VEGF) leading to retinal proliferation and permeability (Aiello, L. P. 2005; Aiello et al., L. P. 1994). Although nearly all patients with diabetes will develop some degree of retinopathy in their lifetime, PDR only occurs in approximately half of these patients. In addition, a high concordance rate (80-90%) between ESRD and PDR has been well documented and underscores a potentially shared underlying pathologic mechanism (Parving, H.-H.2004).

### BRIEF SUMMARY

This disclosure relates to compositions and methods for identifying a subject at risk for developing a microvascular complication associated with diabetes. It is to be understood that both the description of the compositions and methods provided herein is exemplary and explanatory only and is not restrictive of the embodiments as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A shows negative log p-values (Y-axis) from association analyses for 10 SNPs in the chromosome 7q22 region harboring the Epo gene (also see table 3). Black diamonds represent −log p values of all SNPs. Black triangle is from rs1617640 for initial 190 GoKinD type 1 PDR and ESRD cases and 179 controls. Grey square represents rs1617640 for Utah type 2 PDR and ESRD samples. Grey diamond represents all GoKinD type 1 PDR and ESRD samples. The grey circle represents rs1617640 for PDR and ESRD in both GoKinD and Utah type 1 and 2 cohorts. FIG. 1B shows genomic structure and locations of genes between 99.93 M and 100.04 M base pairs (NCBI build 35). Shown is a pairwise D' Hapview plot for SNPs in chromosome 7 region around Epo gene using International HapMap CEU data.

FIG. 2 shows expression of Epo in human and mice. FIG. 2A shows comparison of vitreous levels of Epo in 3 ESRD and PDR patients with a TT genotype and 3 diabetic controls with a GG genotype by an ELISA assay. Each sample was assayed twice. FIG. 2 shows Real Time RT-PCR quantitative analysis of Epo mRNA levels derived from lymphocytes of 13 PDR and ESRD patient blood samples with a TT genotype compared to that of 3 diabetic controls with a GG genotype. FIG. 2C shows Real Time RT-PCR quantitative analysis of Epo mRNA levels derived from kidneys of 3 db/db mice and 2 normal littermate controls. Each RT-PCR was assayed in triplicates. FIG. 2D shows Real Time RT-PCR semiquantitative analysis of Epo mRNA levels derived from retina of 5 mice with oxygen-induced retinal neovascularization (OM model) and 4 control mice. Significance was examined using SPSS's independent samples t-test. The error bars indicate the standard error of the mean.

FIG. 3 shows effects of the rs1617640 variants on luciferase reporter expression in cultured HEK 293 cells. pGL3 luciferase reporter recombinant plasmids containing an Epo promoter sequence with the risk allele T (Promoter (T) construct) or wild-type G allele (Promoter (G) construct) at SNP rs1617640 were transfected in HEK 293 cells. Additionally pGL3 luciferase reporter recombinant plasmids containing a shorter promoter sequence without rs1617640 (Promoter (short)) and the pGL3-Basic vector without insert (Negative control) were transfected in HEK 293 cells. Renilla luciferase plasmid pTK-RL was cotransfected with each construct as an internal control for normalization. Normalized luciferase activity was measured in twelve independent experiments. The mean±SD is given for each construct. Significance was examined using SPSS's independent samples t-test. The error bars indicate the standard error of the mean.

FIG. 4A shows fundus photography of a representative patient with proliferative diabetic retinopathy (PDR) with vitreous hemorrhage (black arrows) and retinal neovascularization (white arrows) from the studied cohort with a TT genotype. FIG. 4B shows histology of diabetic nephropathy. Arrows show areas of mesangial expansion. The white arrow shows a nodular area of mesangial expansion referred to as a Kimmelstiel-Wilson Nodule. The white asterisk shows a region of arteriolar hyalinosis. The black asterisk shows a region of interstitial fibrosis.

FIG. 5 shows graphed −log p-values (Y-axis) from association analyses for the 10 SNPs at the chromosome 7 Epo region. In FIG. 5A, black diamonds represent −log p values of all SNPs around rs1617640. Black triangle represents rs1617640 for initial 190 GoKinD type 1 PDR and ESRD cases and 179 controls. Grey square represents rs1617640 for Utah type 2 PDR and ESRD samples. Grey diamond represents all GoKinD type 1 PDR and ESRD samples. Grey circle represents rs1617640 for PDR in GoKinD and Utah type 1 and 2 combined. FIG. 5B shows LD map (D′) for 10 SNPs surrounding Epo derived from 200 cases in GoKinD cohort. Notice that rs1617640 is in lower overall LD compared to GoKinD controls and Hapmap CEU data shown in FIG. 1.

FIGS. 6A and 6B show Epo immunohistochemistry in human eye Green staining and arrows indicate adventitial expression of Epo in the retinal vasculature of the human eye. Phalloidin, the red staining, labels actin in smooth muscle cells of vasculature. Blue indicates nuclei. Note that the Epo labeling signal is higher in the eye of a normal individual with TT genotype (A), as compared to an eye of normal individual with a GG genotype (B). FIG. 6C shows negative control with the primary antibody pre-absorbed with the Epo antigen. FIG. 6D shows negative control omitting the primary antibody. Scale bars=10 Fm. Abbreviations: ONL, outer nuclear layer; OPL, outer plexiform layer; INL, inner nuclear layer; IPL, inner plexiform layer; GCL, ganglion cell layer.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an oligonucleotide is disclosed and discussed and a number of modifications that can be made to a number of molecules including the oligonucleotide are discussed, each and every combination and permutation of oligonucleotide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. DIABETES

Disclosed herein are compositions and methods for identifying subjects at risk for developing a microvascular complication associated with diabetes mellitus.

The World Health Organization recognizes three main forms of diabetes: type 1, type 2, and gestational diabetes (occurring during pregnancy), which have similar signs, symptoms, and consequences, but different causes and population distributions. Type 1 is usually due to autoimmune destruction of the pancreatic beta cells which produce insulin. Type 2 is characterized by tissue-wide insulin resistance and varies widely; it sometimes progresses to loss of beta cell function. Gestational diabetes is similar to type 2 diabetes, in that it involves insulin resistance. The hormones of pregnancy cause insulin resistance in those women genetically predisposed to developing this condition. Types 1 and 2 are incurable chronic conditions, but have been treatable since insulin became medically available in 1921. Gestational diabetes typically resolves with delivery. Thus, in some aspects of the disclosed method, the subject has been diagnosed with type 1 or type 2 diabetes mellitus or gestational diabetes.

Diabetes can cause many complications. Acute glucose level abnormalities may occur if insulin level is not well-controlled. Serious long-term complications include cardiovascular disease (doubled risk), chronic renal failure (the main cause of dialysis in developed world adults), retinal damage (which can lead to blindness and is the most significant cause of adult blindness in the non-elderly in the developed world), nerve damage (of several kinds), and microvascular damage, which may cause erectile dysfunction (impotence) and poor healing. Poor healing of wounds, particularly of the feet, can lead to gangrene which can require amputation the leading cause of non-traumatic amputation in adults in the developed world.

Diabetes, without qualification, usually refers to diabetes mellitus, but there are several rarer conditions also named diabetes. The most common of these is diabetes insipidus (unquenchable diabetes) in which the urine is not sweet; it can be caused by either kidney (nephrogenic DI) or pituitary gland (central DI) damage.

There are several rare causes of diabetes mellitus that do not fit into type 1, type 2, or gestational diabetes, namely genetic defects in beta cells (autosomal or mitochondrial), genetically-related insulin resistance, with or without lipodystrophy (abnormal body fat deposition), diseases of the pancreas (e.g. chronic pancreatitis, cystic fibrosis), hormonal defects, and chemicals or drugs. In addition, the tenth version of the International Statistical Classification of Diseases (ICD-10) contained a diagnostic entity named “malnutrition-related diabetes mellitus” (MRDM or MMDM, ICD-10 code E12).

The classical triad of diabetes symptoms is polyuria (frequent urination), polydipsia (increased thirst and consequent increased fluid intake) and polyphagia (increased appetite). These symptoms may develop quite fast in type 1, particularly in children (weeks or months) but may be subtle or completely absent—as well as developing much more slowly—in type 2. In type 1 there may also be weight loss (despite normal or increased eating) and irreducible fatigue. These symptoms may also manifest in type 2 diabetes in patients whose diabetes is poorly controlled.

Diabetes mellitus is characterized by recurrent or persistent hyperglycemia, and is diagnosed by demonstrating any one of the following:

fasting plasma glucose level at or above 126 mg/dL (7.0 mmoUL);

plasma glucose at or above 200 mg/dL or 11.1 mmoUL two hours after a 75 g oral glucose load as in a glucose tolerance test;

random plasma glucose at or above 200 mg/dL or 11.1 mmoUL.

Patients with fasting sugars between 6.1 and 7.0 mmoUL (ie, 110 and 125 mg/dL) are considered to have “impaired fasting glucose” and patients with plasma glucose at or above 140 mg/dL or 7.8 mmoUL two hours after a 75 g oral glucose load are considered to have “impaired glucose tolerance.” “Prediabetes” is either impaired fasting glucose or impaired glucose tolerance; the latter in particular is a major risk factor for progression to full-blown diabetes mellitus as well as cardiovascular disease. Thus, in some aspects, the subject has been diagnosed with pre-diabetes.

Thus, in some aspects, the subject has a fasting plasma glucose level of at least 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 mg/dL.

Thus, in some aspects, the subject has a plasma glucose of at least 140, 150, 160, 170, 180, 190, 200, 210, 220, 230, 240, 250, 260, 270, 290 or 300 mg/dL two hours after a 75 g oral glucose load in a glucose tolerance test.

Thus, in some aspects, the subject has a random plasma glucose of at least 140, 150, 160, 170, 180, 190, 200, 210, 220, 230, 240, 250, 260, 270, 290 or 300 mg/dL.

While not generally used for diagnosis, an elevated level of glucose irreversibly bound to hemoglobin (termed glycosylated hemoglobin, Hb_{Alc}, or Al C) of 6.0% or higher (the 2003 revised U.S. standard) is considered abnormal. HbAlc is primarily used as a treatment-tracking test reflecting average blood glucose levels over the preceding 90 days (approximately). However, some physicians may order this test at the time of diagnosis to track changes over time. The current recommended goal for HbAlc in patients with diabetes is <7.0%, which as defined as “good glycemic control”, although some guidelines are stricter (<6.5%). People with diabetes who have HbAlc levels within this range have a significantly lower incidence of complications from diabetes, including retinopathy and diabetic nephropathy.

Thus, in some aspects, the subject has a hemoglobin Hb_{Al}c (Al C) level greater than 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0 percent.

Chronic elevation of blood glucose level leads to damage of blood vessels. In diabetes, the resulting problems are grouped under “microvascular disease” (due to damage to small blood vessels) and “macrovascular disease” (due to damage to the arteries). The damage to small blood vessels leads to a microangiopathy, which can cause diabetic retinopathy and/or diabetic nephropathy. Angiopathy means disease of the blood vessels (arteries, veins, and capillaries). In microangiopathy, the walls of very small blood vessels (capillaries) become so thick and weak that they bleed, leak protein, and slow the flow of blood. For example, diabetics can develop microangiopathy with thickening of capillaries in many areas including the eye.

Diabetic retinopathy refers to growth of friable and poor-quality new blood vessels in the retina as well as macular edema (swelling of the macula), which can lead to severe vision loss or blindness. Retinal damage (e.g., from microangiopathy) makes it the most common cause of blindness among non-elderly adults in the US.

Diabetic nephropathy refers to damage to the kidney which can lead to chronic renal failure, eventually requiring dialysis. Diabetes mellitus is the most common cause of adult kidney failure worldwide in the developed world.

Thus, the microvascular complication of the disclosed method can be nephropathy. The microvascular complication of the method can be retinopathy. Thus, the microvascular complication of the method can be proliferative diabetic retinopathy (PDR).

The disclosed method generally comprises detecting in a sample of nucleic acid from a subject diagnosed with diabetes mellitus a thymidine at position 100155234 of chromosome 7, which is located 1125 bp upstream to the Erythropoietin (Epo) transcription start site.

### B. COMPOSITIONS

1. Erythropoietin

Erythropoietin is a 30,400 M.W. glycoprotein produced primarily in the kidneys in adults and only to a small extent in the liver. The erythropoietin gene is also expressed in the brain and some other tissues, but its physiologic role in these locations has not been established. The gene for erythropoietin is present as a single copy on chromosome 7. The erythropoietin gene is highly conserved on an evolutionary basis with over 90% homology between human and simian genes and an 80% homology between human and rodent erythropoietin genes. The structure and other aspects of erythropoietin are known and are described in, for example, Browne et al., “Erythropoietin: Gene Cloning, Protein Structure, and Biological Properties,” Cold Spring Harbor Symposia on Quantitative Biology, L1, 693-702 (1986); and U.S. Pat. No. 5,106,954 (Fibi et al.) “Erythropoietin Peptides,” which are hereby incorporated by reference at least for material related to the structure of erythropoietin and its gene.

2. Single Nucleotide Polymorphisms (SNP)

The disclosed method generally comprises detecting a single nucleotide polymorphism at position 100155234 of chromosome 7. Specifically, a thymidine rather than a guanine at this position is an indication of increased risk of microvascular complications as disclosed herein.

A Single Nucleotide Polymorphism (SNP) is a DNA sequence variation occurring when a single nucleotide—A, T, C, or G—in the genome (or other shared sequence) differs between members of a species (or between paired chromosomes in an individual). SNPs may fall within coding sequences of genes, noncoding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are non-synonymous. SNPs that are not in protein coding regions may still have consequences for gene splicing, transcription factor binding, or the sequence of non-coding RNA.

3. SNP Detection Methods

A wide variety of techniques have been developed for SNP detection and analysis, see, e.g. Sapolsky et al. (1999) U.S. Pat. No. 5,858,659; Shuber (1997) U.S. Pat. No. 5,633,134; Dahlberg (1998) U.S. Pat. No. 5,719,028; Murigneux (1998) WO98/30717; Shuber (1997) WO97/10366; Murphy et al. (1998) WO98/44157; Lander et al. (1998) WO98/20165; Goelet et al. (1995) WO95/12607 and Cronin et al. (1998) WO98/30883. In addition, ligase based methods are described by Barany et al. (1997) WO97/31256 and Chen et al. Genome Res. 1998; 8(5):549-56; mass-spectroscopy-based methods by Monforte (1998) WO98/12355, Turano et al. (1998) WO98/14616 and Ross et al. (1997) Anal Chem. 15, 4197-202; PCR-based methods by Hauser, et al. (1998) Plant J. 16, 117-25; exonuclease-based methods by Mundy U.S. Pat. No. 4,656,127; dideoxynucleotide-based methods by Cohen et al. WO91/02087; Genetic Bit Analysis or GBA by Goelet et al. WO92/15712; Oligonucleotide Ligation Assays or OLAs by Landegren et al. (1988) Science 241:1077-1080 and Nickerson et al. (1990) Proc. Natl. Acad. Sci. (U.S.A.) 87:8923-8927; and primer-guided nucleotide incorporation procedures by Prezant et al. (1992) Hum. Mutat. 1:159-164; Ugozzoli et al. (1992) GATA 9:107-112; Nyreen et al. (1993) Anal. Biochem. 208:171-175, which are all hereby incorporated herein by reference for the teaching of SNP detection methods.

The disclosed method contemplates the use of any discovered method of detecting the disclosed. For example, the method can comprise the use of restriction fragment length polymorphism; allele specific hybridization; TagMan®, Molecular Beacon, or Scorpion® assay; allele specific oligonucleotide ligation; invader method; rolling circle DNA amplification; mass spectroscopy; gene sequencing, or variations thereof.

i. Allele Specific Hybridization

The provided method can comprise detecting the SNP by Allele Specific Hybridization. This method relies on selective hybridization to distinguish between two DNA molecules differing by one base. In general, the method involves applying labeled PCR fragments to immobilized oligonucleotides representing SNP sequences. After stringent hybridization and washing conditions, label intensity is measured for each SNP oligonucleotide.

Thus, the provided method can comprise providing a nucleic acid probe that hybridizes under stringent conditions to an oligonucleotide consisting of SEQ ID NO:1 but does not hybridize under stringent conditions to an oligonucleotide consisting of SEQ ID NO:2, and detecting hybridization of said probe to the nucleic acid sample. The nucleic acid probe can comprise at least about 5, 6, 7, 8, 9, 10, 11, 12, 13, 14, 15, 16, 17, 18, 19, 20, 21, 22, 23, 24, 25, 26, 27, 28, 29, or 30 nucleotides.

The probe can comprise a label such as a fluorescent dye (also known herein as fluorochromes and fluorophores). Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAH); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350^{TH}; Alexa Fluor 430^{TH}; Alexa Fluor 488^{TH}; Alexa Fluor 532^{TM}; Alexa Fluor 546^{TH}; Alexa Fluor 568^{TH}; Alexa Fluor 594^{TH}; Alexa Fluor 633^{TM}; Alexa Fluor 647^{TH}; Alexa Fluor 660^{TH}; Alexa Fluor 680^{TH}; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG^{TM} CBQCA; ATTO-TAG^{TM} FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO^{TM}-1; BOBO^{TM}-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO^{TM}-1; BOPRO^{TM}-3; Brilliant Suiphoflavin FF; BTC; BTC-5N; Calcein; Calcein Blue; Calcium Crimson −; Calcium Green; Calcium Green-1 Ca^{t+} Dye; Calcium Green-2 Ca^{t+}; Calcium Green-5N Ca²⁺; Calcium Green-C18 Ca^{t+}; Calcium Orange; Calcofluor White; Carboxy-X-rhodamine (5-ROX); Cascade Blue^{TM}; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NEU; CMFDA; Coelenterazine; Coelenterazine cp; Coelenterazine f; Coelenterazine fep; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2^{TM}; Cy3.1 8; Cy3.5^{TM}; Cy3^{TM}; Cy5.1 8; Cy5.5^{TM}; Cy5^{TM}; Cy7^{TM}; Cyan GFP; cylic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-APPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC 18(3)); I Dinitrophenol; DiO (DiOC18(3)); DiR; DiR (DilC 18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (Et-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; FluoroGold (Hydroxystilbamidine); Fluor-Ruby; Fluor X; FM 1-43^{TM}; FM 4-46; Fura Red^{TM} (high pH); Fura Red^{TM}/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAP; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green^{TM}; Oregon Green^{TM} 488; Oregon Green^{TM} 500; Oregon Green^{TM} 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP^{TM} (super glow BFP); sgGFP^{TM} (super glow GFP); SITS (Primulin; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red^{TM}; Texas Red-x^{TM} conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

ii. Single-Step Homogeneous Methods

TagMan®, molecular beacon, and Scorpion® assay are all microtiter plate-based fluorescent readout systems, initially designed for real time PCR expression analyses. TagMan® and molecular beacon both rely on allele-specific hybridization of oligonucleotides during PCR for allele discrimination, while scorpion assay can use either allele-specific PCR or allele-specific hybridization chemistry for allelic discrimination. They all can be performed as an endpoint assay in a completely homogeneous reaction. All the reagents and genomic DNA are mixed at the beginning, and the fluorescent signal is read after the thermocycling step. There is no separate pre-amplification step, or intermediate processing, making them the simplest assay formats possible.

Thus, the provided method can comprise detecting the SNP using TagMan®. Allelic discrimination using this chemistry is based on the design of two TagMan® probes, specific for the wildtype allele and the mutant allele. TagMan® SNP analysis utilizes the 5′ exonuclease activity of DNA Taq polymerase and the quenching effects of specific florescent dyes to determine the relative frequency of each allele within an individual genome. Primers are designed against a conserved region of the genome flanking the locus of interest. Two probes are designed across the locus of interest, one for each allele. Each probe is labeled with a different reporter dye as well as a quencher molecule. Proximity to the quencher dye inhibits the florescence of the reporter molecule. During thermocycling, the probe anneals to the locus of interest in an allele specific manner. As the Taq DNA polymerase extends the primers, it also degrades the annealed probe, allowing the florescent dye to come out of the sphere of influence of the quencher and thus become detectable.

The provided method can comprise detecting the SNP using molecular beacons. Molecular beacons are oligonucleotide probes that can report the presence of specific nucleic acids in homogenous solutions (Tyagi S, Kramer F R. Molecular beacons: probes that fluoresce upon hybridization, Nature Biotechnology 1996; 14: 303-308). Molecular beacons are hairpin shaped molecules with an internally quenched fluorophore whose fluorescence is restored when they bind to a target nucleic acid. They are designed in such a way that the loop portion of the molecule is a probe sequence complementary to a target nucleic acid molecule. The stem is formed by the annealing of complementary arm sequences on the ends of the probe sequence. A fluorescent moiety is attached to the end of one arm and a quenching moiety is attached to the end of the other arm. The stem keeps these two moieties in close proximity to each other, causing the fluorescence of the fluorophore to be quenched by energy transfer. Since the quencher moiety is a non-fluorescent chromophore and emits the energy that it receives from the fluorophore as heat, the probe is unable to fluoresce. When the probe encounters a target molecule, it forms a hybrid that is longer and more stable than the stem and its rigidity and length preclude the simultaneous existence of the stem hybrid. Thus, the molecular beacon undergoes a spontaneous conformational reorganization that forces the stem apart, and causes the fluorophore and the quencher to move away from each other, leading to the restoration of fluorescence.

The provided method can comprise detecting the SNP using Scorpion® primers. Scorpion® primers are bi-functional molecules in which a primer is covalently linked to the probe. The molecules also contain a fluorophore and a quencher. In the absence of the target, the quencher nearly absorbs the fluorescence emitted by the fluorophore. During the Scorpion® PCR reaction, in the presence of the target, the fluorophore and the quencher separate which leads to an increase in the fluorescence emitted. The fluorescence can be detected and measured in the reaction tube. The Scorpion® primer carries a Scorpion® probe element at the 5′ end. The probe is a self-complementary stem sequence with a fluorophore at one end and a quencher at the other. The Scorpion® primer sequence is modified at the 5′ end. It contains a PCR blocker at the start of the hairpin loop (Usually HEG monomers are added as blocking agent). In the initial PCR cycles, the primer hybridizes to the target and extension occurs due to the action of polymerase. Scorpion® primers can be used to examine and identify point mutations by using multiple probes. Each probe can be tagged with a different fluorophore to produce different colors. In Scorpion® primers, the probe is physically coupled to the primer which means that the reaction leading to signal generation is a unimolecular one. This is in contrast to the bi-molecular collisions required by other technologies such as TagMan© or Molecular Beacons. After one cycle of PCR extension completes, the newly synthesized target region will be attached to the same strand as the probe. Following the second cycle of denaturation and annealing, the probe and the target hybridize. The denaturation of the hairpin loop requires less energy than the new DNA duplex produced. Consequently, the hairpin sequence hybridizes to a part of the newly produced PCR product. This results in the separation of the fluorophore from the quencher and causes emission.

The provided method can comprise detecting the SNP using an allele-specific amplification primers that have secondary priming sites for universal energy-transfer-labeled primers.

The provided method can comprise detecting the SNP using an AlphaScreen proximity assay. AlphaScreen generates an amplified light signal when donor and acceptor beads are brought to proximity, and this detection method can be combined with allele-specific amplification chemistry or allele-specific hybridization chemistry for allele discrimination.

iii. Allele Specific Oligonucleotide Ligation

The provided method can comprise detecting the SNP by Allele Specific Oligonucleotide Ligation. By designing oligonucleotides complementary to the target sequence, with the allele-specific base at its 3′-end or 5-′end, one can determine the genotype of the PCR amplified target sequence by determining whether an oligonucleotide complementary to the DNA sequencing adjoining the polymorphic site is ligated to the allele-specific oligonucleotide or not.

iv. Invader Method

There have been a few notable efforts to establish PCR-free genotyping methods. One such attempt is the Invader method (Third Wave Technologies), based on a matched nucleotide-specific cleavage by a structure-specific ‘flap’ endonuclease, in the presence of an invading oligonucleotide. The combination of this reaction with a secondary reaction using fluorescence resonance energy transfer (FRET) oligonucleotide cassettes, generates a highly allele-specific signal, in a completely homogeneous and isothermal reaction. In addition, the Invader assay's great sensitivity and excellent signal to noise ratio allow direct genotyping of genomic DNA samples without PCR. However, the amount of DNA currently required for reliable genotyping is high (50 ng range) for the analysis of a large number of SNPs. The Invader method can be combined with PCR to reduce the DNA requirement, which also makes the signal more robust.

v. Rolling Circle DNA Amplification

Another type of PCR-free genotyping is available through the combination of padlock probe ligation, and signal amplification by the rolling circle DNA amplification (RCA) process. In this assay, allele discrimination is accomplished by the specific ligation of completely matched oligonucleotides, in the same way as oligonucleotide ligation assay (OLA). The difference here is that the ligation of a padlock probe creates a circular DNA, which can be amplified by rolling circle DNA synthesis by a DNA polymerase. The high degree of signal amplification by rolling circle synthesis and the specificity of the allele-discrimination by DNA ligase, make padlock probe/RCA assay sensitive enough to be directly applied to genomic DNA. However, typical padlock probe/RCA genotyping still requires a large quantity of DNA (100 ng) per genotype, again making it less than ideal for the analysis of many SNPs. However, FRET primers (Amplifluor) can be used for signal detection in reducing the DNA requirement to a nanogram level.

vi. Mass Spectroscopy

The provided method can comprise detecting the SNP by mass spectroscopy. The principle of this method is to use mass spectrometry to detect the product of enzymatic allele-discrimination reaction directly or indirectly. Various allele discrimination chemistries such as single-base extension and its variation, allele-specific hybridization of peptide nucleic acid (PNA), Invader, and allele-specific PCR, have all been successfully combined with the mass spectrometry detection. Combinations of single-base extension or its modifications with matrix-assisted laser desorption/ionization time-of-flight (MALDI-TOF) mass spectrometry are the most commonly used, and have been made into commercial products by companies such as Sequenom and Applied Biosystems/PerSeptive Biosystems. The advantage of the MALDI-TOF mass spectrometry-based detection is in its speed and multiplexing capability. For example, a moderate mass spectrometer capable of recording 40,000 spectra a day, can theoretically score 200,000 genotypes in a 5-plex detection format. However, their rate limiting steps are generally not in the detection process by a mass spectrometer, but are in the preceding enzymatic reactions, and post-reaction sample processing steps. In most mass spectrometry-based assays, 5-plex may be the realistic limit for multiplexing to get reliable signals, partly due to the limitations in the detectable mass range and in the sensitivity of mass discrimination. Post-reaction sample processing is more complicated than that of most other genotyping formats, as a very high purity is necessary for the samples to be analyzed by a mass spectrometer. Solid phase sample processing with ion-exchange resin is employed in Sequenom's MassArray automated system, while miniaturized reverse phase liquid chromatography is used for Applied Biosystems/PerSeptive Biosystem's product to address this issue. Another system called ‘GOOD assay’ involves a use of chemically modified primers in the reaction, followed by an enzymatic removal of unextended primers and alkylation of the product, allowing a simplified and effective sample preparation for mass spectrometry.

Genotype accuracy due to the intrinsic nature of mass spectrometry is another advantage. The sensitivity of the instrument, the mass specificity of each reaction product, and for some type of reactions the fact that each reaction contains internal standards for calibration, all contribute to this accuracy. Mass spectrometry-based methods give little background especially when detecting the allelic discrimination reaction products directly, allowing accurate and automated genotype calling.

A different mass spectrometry-based assay has been made into a commercial product as Qiagen's MassCode system. This assay combines allele-specific PCR with UV-cleavable ‘mass tags’, and mass spectrometry detection. Here, mass spectrometry detects the cleaved tags and not the extension products themselves. Use of these ‘mass tags’ makes highly-multiplexed detection by a relatively simple mass spectrometer possible. One the other hand, this method can be more prone to background signal at least theoretically, as the mass spectrometer does not directly detect the allele-discrimination reaction product. For example, incomplete removal of free ‘mass tag’ labeled primers before UV-cleavage can cause a false signal in this method.

Matrix-assisted laser desorption ionization (MALDI) is a soft ionization technique used in mass spectrometry, allowing, among other things, the ionization of biomolecules (biopolymers such as proteins, peptides and sugars) which tend to be more fragile and quickly lose structure when ionized by more conventional ionization methods. It is most similar in character to electrospray ionization both in relative softness and ions produced. The ionization is triggered by a laser beam (normally a nitrogen-laser). A matrix is used to protect the biomolecule from being destroyed by direct laser beam.

vii. Sequencing

The provided method can comprise detecting the SNP by gene sequencing. Sequencing is the procedure of choice for SNP discovery. The most common forms of sequencing are based on primer extension using either a) dye-primers and unlabeled terminators or b) unlabeled primers and dye-terminators. The products of the reaction are then separated using electrophoresis using either capillary electrophoresis or slab gels.

Pyrosequencing employs an elegant cascade of enzymatic reactions to detect nucleotide incorporation during DNA synthesis. When a nucleotide is incorporated at the 3′-end by DNA polymerase, a pyrophosphate is released that is immediately converted to ATP by ATP sulfurylase. This ATP causes the oxidization of luciferin by luciferase, which is detected as a light signal. Pyrosequencing was initially developed as a DNA sequencing method, with a chemistry completely different from the Sanger dideoxynucleotide method. It is also a unique homogeneous sequencing method with no electrophoresis. Its capability to read flanking sequences as well as the SNP position itself, and its high specificity (i.e. non-specific binding will not generate a false signal) make it an accurate and attractive SNP genotyping method. In this method, alleles can be called by analyzing the individual sample itself, without comparing its signal to that of other samples or controls. This makes Pyrosequencing suitable for fully automated genotype calling, an important component of high throughput analyses. A 96-well medium throughput machine and a fully automated 384-well format high-throughput machine, are available from Pyrosequencing AB (Uppsala, Sweden) for this method, and the latter has capacity to score high thousands to low tens of thousands of genotypes a day. Pyrosequencing can be done in a duplex or a triplex format at least for some SNP combinations.

4. Primers and Probes

Thus, disclosed are compositions including primers and probes, which are capable of interacting with the disclosed nucleic acids, such as those comprising the SNP disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

5. Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

### C. METHODS OF MONITORING AND TREATMENT

Also provided is a method of monitoring a subject identified as at increased risk for a microvascular complication by the methods disclosed herein. As disclosed herein, the disclosed SNP is located 1125 nucleotide base pairs upstream from the Erythropoietin (Epo) transcription start site and affects gene transcription. Further as disclosed herein, the increased levels of Epo resulting from the SNP is involved in the development of microvascular complications. Thus, for example, the subject can be monitored for levels of erythropoietin in a tissue or bodily fluid. For example, the method can comprise obtaining a tissue or bodily fluid from a subject identified as having the disclosed SNP and measuring Epo levels in the bodily fluid. The bodily fluid can be, for example, blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. The tissue can be any tissue that suffers microvascular complications associated with diabetes. Thus, the tissue can be obtained from, for example, the kidney or eye.

It is understood that any discovered method for the detection and measurement of Epo levels can be used in the disclosed method. For example, an immunodetection method can be used. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, flow cytometry, protein arrays, multiplexed bead arrays, magnetic capture, in vivo imaging, fluorescence resonance energy transfer (FRET), and fluorescence recovery/localization after photobleaching (FRAP/FLAP).

In addition, or alternatively, the subject can be monitored for signs of microvascular complications. For example, the subject can be monitored for neovascularization in susceptible tissues, such as, for example, the retina. This can involve a detailed eye examination. For example, Early Treatment Diabetic Retinopathy Study (ETDRS) protocol seven standard field stereoscopic fundus photography and fluorescein angiography can be used to monitor a subject for signs of diabetic retinopathy. The subject can be graded according to clinical ETDRS criteria (no retinopathy, mild nonproliferative diabetic retinopathy (NPDR), severe NPDR, very severe NPDR, PDR less than high risk, and PDR with high risk characteristics).

Also provided is a method of choosing a treatment protocol for a subject identified as at increased risk for a microvascular complication by the methods disclosed herein. For example, a more aggressive treatment protocol can be recommended for a subject identified as having the disclosed SNP. Examples of more aggressive treatments include, for example, glucose control and diabetic intensive intervention.

In addition, a protocol for treatment of a subject identified as at risk for or as having a microvascular complication by the methods disclosed herein can involve the inhibition of Epo in the subject. Thus, disclosed is a method of treating a subject identified as having the disclosed SNP, comprising administering to the subject a therapeutically effective amount of an erythropoietin inhibitor. For example, an Epo inhibitor can be administered to the subject by intraocular delivery to prevent diabetic retinopathy.

1. Epo Inhibitors

The Epo inhibitor can be an antagonist of an activity of Epo. “Activities” of a protein include, for example, transcription, translation, intracellular translocation, secretion, phosphorylation by kinases, cleavage by proteases, homophilic and heterophilic binding to other proteins, ubiquitination.

The Epo antagonist can be a small molecule, soluble receptor, antibody, functional nucleic acid (e.g., siRNA, antisense), dominant negative form of Epo, or a dominant negative form of the Epo receptor. In addition, the Epo inhibitor can be an antagonist of Epo signaling. Thus, the method can comprise administering to the subject an inhibitor of the JAKISTAT pathway.

Examples of erythropoietin antagonists include, but are not limit to the erythropoietin mutant R103A (Burns et al., Blood. 2002 Jun. 15; 99(12):4400-5), the EPO mimetics EMP and EMP33 (Johnson et al., Biochemistry, 1998, 37, 3699-1710; Livnah et al., Nat Struct Biol. 1998, 5(11):993-1004; Yasuda et al., Carcinogenesis 2003, 24:1021-1029), anti-EPO antibodies (Yasuda et al., Carcinogenesis. 2002, 23(11):1797-805), and the soluble form of the EPO receptor (Yasuda et al., Carcinogenesis. 2002, 23(11):1797-805).

i. Soluble Receptor

The Epo inhibitor can be polypeptide that binds Epo and acts as a competitive inhibitor. For example, the Epo inhibitor can be a soluble form of the Epo receptor.

The amino acid sequence of full length human Epo receptor is found in SEQ ID NO: 100 (Accession No. AAB23271). The corresponding nucleotide sequence of full length human Epo receptor is found in SEQ ID NO: 101. Thus, the Epo inhibitor can be a polypeptide comprising a fragment of SEQ ID NO:100 of at least about 4, 5, 6, 7, 8, 9, 10, 15, 20, 25, 30, 35, 40 amino acids in length that can bind Epo without activating signal transduction.

The extracellular domain of Epo receptor is from amino acid 1 to 251 Leu, which can be used for inhibiting Epo signal transduction. For example, a recombinant ligand-binding domain of human EPO-receptor is provided is U.S. Pat. No. 5,378,808 and also described in Yet M G, Jones S S, Blood, 1993 Sep. 15; 82(6):1713-9, which are hereby incorporated herein by reference for their teaching of these soluble peptides and methods of making and using same.

In addition, the Epo inhibitor can be a non-functional fragment of Epo that can bind the Epo receptor without activating signal transduction. The amino acid sequence of full length human erythropoietin is found in SEQ ID NO: 3 (Accession No. X02158). The corresponding nucleotide sequence of full length human erythropoietin is found in SEQ ID NO: 4. Thus, the Epo inhibitor can be a polypeptide comprising a fragment of SEQ ID NO:3 of at least about 4, 5, 6, 7, 8, 9, 10, 15, 20, 25, 30, 35, 40 amino acids in length that can bind the Epo receptor without activating signal transduction.

Other such competitive inhibitors can be designed based on known binding sites of ligand, receptor, signaling molecules, and/or target substrates.

For example, a soluble form of the EPOR has been used to probe a phage display library to identify candidate peptides having EPO-like properties. Wrighton et al., in Science 273:458 (1996) described the use of a fusion protein comprising the EPOR extracellular domain and human placental alkaline phosphatase in a library screening protocol. Peptides having higher affinity for the EPOR were subsequently isolated from mutagenesis libraries that displayed plll protein fusions. This approach led to the identification of several peptides that stimulated erythropoiesis in mice. As another example, U.S. Pat. No. 6,642,353 provides a peptide capable of binding to a human erythropoietin receptor, which is hereby incorporated herein by reference for its teaching of these peptides and methods of making and using same.

ii. Antibodies

The Epo inhibitor can be an antibody. For example, antibodies specific for one or more of ligand, receptor, signaling molecules, and/or target substrates can be generated and used to inhibit Epo activity. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with Epo such that Epo is inhibited from interacting with the Epo receptor. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fe fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Administration of the antibodies can be done as disclosed herein. Nucleic acid approaches for antibody delivery also exist. The broadly neutralizing antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

iii. Functional Nucleic Acids

The Epo inhibitor of the provided method can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Epo or the genomic DNA of Epo or they can interact with the polypeptide Epo. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10″⁶, 10₈, 10⁻¹⁰, or 10₁₂. A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically atamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with Kₐ's from the target molecule of less than 10-12 M. It is preferred that the aptamers bind the target molecule with a Kd less than 10⁶, 10⁻⁸, 10″₁₀, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a Kd with the target molecule at least 10, 100, 1000, 10,000, or 100,000 fold lower than the Kd with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10-6, 10-8, 10-10, or 10-12. Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNAse P, which cleaves the target molecule. EGSs can be designed to specifically target a RNA molecule of choice. RNase P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et at. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for Epo.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

Thus, the Epo inhibitor of the provided method can be a functional nucleic acid comprising a fragment of SEQ ID NO:101, or the complement thereof, of at least about 4, 5, 6, 7, 8, 9, 10, 15, 20, 25, 30, 35, 40 nucleic acids in length that can bind Epo Receptor mRNA, DNA, or protein.

Thus, the Epo inhibitor of the provided method can be a functional nucleic acid comprising a fragment of SEQ ID NO:4, or the complement thereof, of at least about 4, 5, 5, 7, 8, 9, 10, 15, 20, 25, 30, 35, 40 nucleic acids in length that can bind the Epo mRNA, DNA, or protein.

2. Pharmaceutical Carriers

The disclosed compositions can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

3. Administration

A composition disclosed herein may be administered in a number of ways depending on whether local or systemic treatment is desired, and on the area to be treated. For example, the compositions may be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via incubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of the Epo inhibitor used alone might range from about 1 μg/kg to up to 100 mg/kg of body weight or more per day, depending on the factors mentioned above.

Following administration of a disclosed composition for treating, inhibiting, or preventing microvascular complications associated with diabetes, the efficacy of the therapeutic can be assessed in various ways well known to the skilled practitioner. For instance, one of ordinary skill in the art will understand that a composition disclosed herein is efficacious in treating or inhibiting a microvascular complications in a subject by observing that the composition inhibits Epo activity, reduces circulating or localized concentrations of Epo or prevents a further increase in circulating or localized concentrations of Epo. Epo concentrations can be measured by methods that are known in the art, for example, using polymerase chain reaction assays to detect the presence of Epo mRNA or antibody assays to detect the presence of Epo protein in a sample (e.g., but not limited to, blood) from a subject or patient, or by measuring the level of circulating Epo levels in the patient. Efficacy of the administration of the disclosed composition may also be determined by measuring neovascularization in, for example, the retina of the subject.

The compositions that inhibit Epo may be administered prophylactically to patients or subjects who are at risk for microvascular complications.

The disclosed compositions and methods can also be used for example as tools to isolate and test new drug candidates for a variety of diabetes related diseases/complications.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### E. Definitions

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a oligonucleotide” includes a plurality of such oligonucleotides, reference to “the oligonucleotide” is a reference to one or more oligonucleotides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value foluis another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “effective amount” refers to such amount as is capable of performing the function of the compound or property for which an effective amount is expressed. As will be pointed out below, the exact amount required will vary from process to process, depending on recognized variables such as the compounds employed and the processing conditions observed. Thus, it is not typically possible to specify an exact “effective amount.” However, an appropriate effective amount may be determined by one of ordinary skill in the art using only routine experimentation. In various aspects, an amount can be therapeutically effective; that is, effective to treat an existing disease or condition. In further various aspects, a preparation can be prophylactically effective; that is, effective for prevention of a disease or condition. In a further aspect, a compound or moiety can be provided in an amount effective to perform an imaging function.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Promoter Polymorphism in the Erythropoietin Gene and Severe Diabetic Microvascular Complications

i. Materials and Methods

Patients:

This study was approved by the GoKinD Access Committee and the University of Utah Institutional Review Board. All subjects provided informed consent prior to participation in the study. The GoKinD (Genetics of Kidneys in Diabetes) study is a collection of North American probands with type I diabetes (T1DM), with and without diabetic nephropathy. Details of the GoKinD study are available at the GoKinD website and are published (Mueller P. W. et al., 2006). The Utah ESRD patients were diagnosed and enrolled at renal dialysis facilities of the University of Utah and have also undergone detailed eye examination, ETDRS protocol seven standard field stereoscopic fundus photography and fluorescein angiography at the Moran Eye Center of University of Utah. Retinopathy status was determined by evaluation of the fundus photographs and graded according to clinical ETDRS criteria (no retinopathy, mild nonproliferative diabetic retinopathy (NPDR), severe NPDR, very severe NPDR, PDR less than high risk, and PDR with high risk characteristics). Patients with any neovascularization of disc, neovascularization elsewhere, vitreous hems, fibrovascular proliferation, or tractional retinal detachment were considered part of the PDR cohort.

Retinopathy grading was performed without knowledge of the genetic phenotype. Diabetic controls (individuals with diabetes for 10 years or longer duration without any nephropathy or retinopathy) were enrolled at the Utah Diabetic Center and Moran EyeCenter. A subject was considered to have definite diabetic nephropathy if he/she had ESRD (Stage V chronic kidney disease on dialysis or renal transplantation). The subjects evaluated in the study had both ESRD and a history of PDR. Patient characteristics of GoKinD and Utah cohorts are listed in Table 1.

Genotyping:

The initial GoKinD cohort of 369 Caucasian T1DM patients, including 190 individuals with both PDR and ESRD, was genotyped and allele frequencies were compared to 179 ethnicity matched diabetic control patients. The expanded GoKinD samples for second stage replication genotyping of rs1617640 included a total of 320 T1DM patients with both ESRD and PDR, and 449 T1DM diabetic control patients (Mueller P. W. et al., 2006). In addition, a third Utah cohort of 151 Caucasian T2DM patients including 70 with both PDR and ESRD and 81 ethnicity matched diabetic control patients, was genotyped.

For SNP genotyping, genomic DNA extracted were PCR-amplified from case and control patients' blood samples. Twenty-eight SNPs were genotyped using the SNAPSHOT method on an ABI 3130 genetic analyzer (Applied Biosystems, Foster City, Calif.) as previous described (Yang Z. et al., 2006) and according to the manufacturer's instructions. 19 tagging SNPs in candidate genes involved in angiogenesis were chosen based on the International HapMap CEU data. A complete list of primers for each gene used in the SNP SNPSHOT assay is listed in Table 2.

Data Analysis:

The chi-squared test for trend for the additive model over alleles was performed to assess evidence for association using PEP1 version 4.0 (Abramson, J. H. and Gahlinger, P. M. 2001). Odds ratios and 95% confidence intervals were calculated by conditional logistic regression using SPSS version 13.0 to estimate risk size for the heterozygotes and homozygotes for the risk alleles. For the risk genotypes identified, population attributable risks (PAR) were calculated using the Levin formula (Levin, M. L. 1953). LD structure was examined using Haploview (version 3.32) (Barrett, J. C., et al. 2005). The default settings were used creating a 95% confidence bounds on D′ to define SNP's in strong LD (Gabriel S. B. et al., 2002).

ELISA Assay for Human Epo Vitreous Level:

Undiluted human vitreous samples were obtained at time of pars plana vitrectomy surgery. Concentration of Epo in human vitreous was measured by sandwich ELISA. Polyclonal anti-Epo antibody (R&D Systems, Minneapolis, Minn.) was coated on PVC microtiter wells in carbonate/bicarbonate buffer (pH9.6, 0.05M) at 4° C. overnight. After washing, the remaining protein-binding sites were blocked by 10% goat serum at room temperature for 2 hours. Twenty five Fl vitreous samples, or Epo standard sample (Cell Sciences, Canton, Mass.) at different concentrations, and 75 Fl 10% goat serum were added to each well, and the plate was placed on a horizontal shaker at room temperature for 1 hour. After washing, monoclonal anti-Epo antibody (R&D Systems, Minneapolis, Minn.) and HRP conjugated goat anti-mouse IgG antibody (Santa Cruz, Santa Cruz, Calif.) were added consequently. Finally, TMB solution was added to develop color and the absorbance was read at 655 nm by a Benchmark Plus microplate reader (BIO-RAD, Hercules, Calif.). Concentration of Epo was calculated from standard curve.

Epo Immunohistochemistry:

Human donor eyes were obtained from Utah Lions Eye Bank. Cryosections were obtained after fixation with 4% paraformaldehyde on ice for 2 hours, and incubated in 15% and 30% sucrose overnight consequently as previously described (Yang Z. et al., 2006). Epo signaling was labeled using 2 Fg/ml monoclonal anti-Epo antibody (R&D Systems, Minneapolis, Minn.) and goat anti-mouse IgG secondary antibody (Jackson Immunoresearch, West Grove, Pa.). To test the specificity of the antibody, some sections were labeled with an anti-Epo antibody, preabsorbed with recombinant human Epo protein in a 1:16 molar ratio (Cell Sciences, Canton, Mass.). Phalloidin (Molecular Probes, Carlsbad, Calif.) and TO-PRO-3 iodide (Molecular Probes, Carlsbad, Calif.) were used to label actin and the nuclei, respectively Immunolabeling was visualized using a Zeiss LSM 510 laser scanning confocal microscope (Zeiss, Thomwood, N.Y.) at identical parameters.

Relative Quantitation of Epo mRNA in Human Lymphocytes:

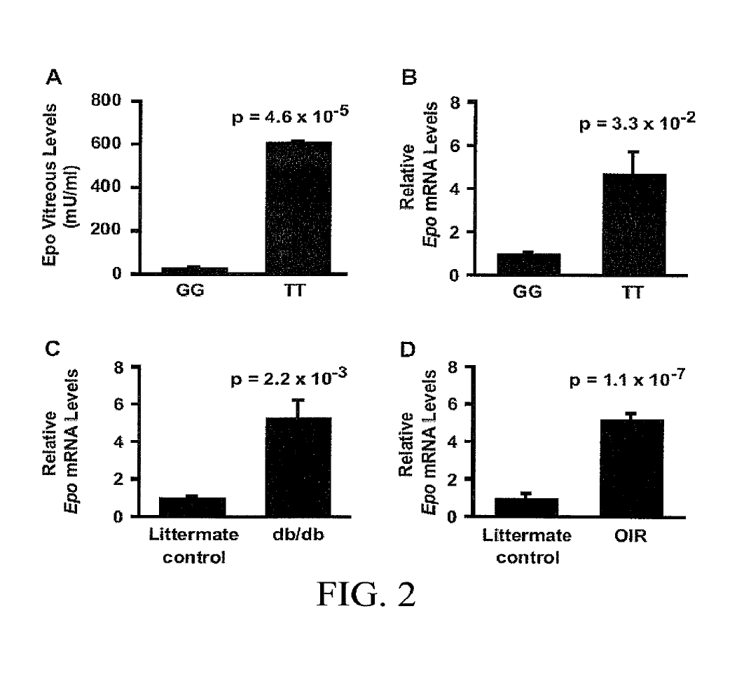

The Epo mRNA level in lymphocytes derived from study subjects was studied using TagMan® Gene Expression Assays (Applied Biosystems: Hs01071097_ml). RNA was extracted from patient's lymphocytes from peripheral blood samples and converted into cDNA (Invitrogen, SuperScrit™ III First-Strand Synthesise System for RT-PCR, Cat. No: 18080-051). Real-time PCR was performed on an ABI 7300 real-time PCR system. GAPDH was used as an endogenous control (Applied Biosystems: 4326317E). Epo and GAPDH standard curves had similar amplification kinetics. RNA, from diabetic control patients with GG genotype and 16 PDR and ESRD patients with a TT genotype, was assayed in duplicate reactions on 3 different runs. Expression of GAPDH was used to normalize the Epo expression. Results are presented as percent increase in Epo mRNA levels for TT samples relative to GG samples. GAPDH normalized Epo expression in the TT genotype is 6 fold greater than that of the GG genotype (FIG. 2b).

Oxygen-Induced Retinopathy and Db/Db Mouse Models:

Oxygen-induced retinopathy (OIR) model Retinal neovascularization was produced in C57BL/6 mice by placing postnatal day 7 (P7) mice and their mothers in an atmosphere of 75±3% oxygen for 5 days (Smith L. E. et al., 1994). Oxygen concentration was automatically monitored and controlled by an oxygen controller (BioSpherix, Redfield, N.Y.). At P12, the mice were returned to room air for 5 days. At P17, mice were sacrificed, their eyes were rapidly removed and dissected. The retina was used for RT-PCR study to measure in RNA level of Epo.

Diabetic Kidney Disease in the Db/Db Mouse Model:

The db/db mice are among the best characterized and most intensively investigated mouse model for diabetic kidney disease (Sharma, K., et al. 2003). Animals were obtained from Jackson Laboratory.

RT-PCR of Epo mRNA in Mouse Retina and Kidney:

Total RNA was isolated from retinas of the OIR mice described above or from 15 week db/db mice. The RNA was converted into cDNA (Invitrogen, SuperScrit™ III First-Strand Synthesise System for RT-PCR, Cat. No: 18080-051). 50 ng cDNA was used for real-time PCR (Qiagen, QuantiTect SYBR Green PCR Kit) with the following primers for the mouse Epo gene to generate a 166 bp product:

(SEQ ID NO: 5)
Mus-Epo-qPCR-L: ccaccctgctgcttttactc,
and
(SEQ ID NO: 6)
Mus-Epo-qPCR-R: ctcagtctgggaccttctgc.

The following primers for GAPDH expression were used to normalize Epo expression:

(SEQ ID NO: 7)
Mus-GAPDHQPCR-L2: gtgaaggtcggtgtgaacgg,
and
(SEQ ID NO: 8)
Mus-GAPDH-QPCR-R2: gccgttgaatttgccgtgag.

The RT-PCR was performed simultaneously for Epo and GAPDH on an ABI 7300 real-time PCR system. RT-PCR conditions were one cycle of 50° C.-2 minutes, 95° C.-15 minutes, followed by 35 cycles in which each cycle included 94° C.-15 seconds, 58° C.-30 seconds and 72° C.-30 seconds.

Expression Constructs and Luciferase Reporter Assays:

A DNA fragment containing −1357 to +180 bp from the Epo transcription site including either the G (Promoter (G) construct) or the T allele (Promoter (T) construct) of rs1617640 at −1125 was PCR amplified from genomic DNA of diabetic patients using the following primers:

(SEQ ID NO: 9)
forward: cggggtaccaactcctgggctcaaaggat,
and
(SEQ ID NO: 10)
reverse: ccgctcgagtccgcgcctggccggggtccctcag.

A DNA fragment containing −963 to +180 bp from Epo transcription site without rs1617640 (Promoter (short)) was PCR amplified from genomic DNA of diabetic patients using the following primers:

(SEQ ID NO: 11)
forward: cggggtacccctgcacaatggactgtgtgctctg,
and
(SEQ ID NO: 12)
reverse: ccgctcgagtccgcgcctggccggggtccctcag.

These constructs were subcloned into the Kpn I-Xho I site of the pGL3-basic vector (Promega, Madison, Wis., USA) respectively. All constructs were verified by restriction digestion and DNA sequencing. Positive control plasmid (pGL3-Control Vector) contains SV40 enhancer and promoter driving luciferase reporter was obtained from Promega (Madison, Wis., USA). HEK 293 cells were split into 24-well plates and cotransfected 24 hr later with 1 ng of the transfection control Renilla luciferase plasmid pTK-RL (Promega, Madison, Wis., USA) and 200 ng of one of the following plasmids: pGL3-control plasmid, promoter (short) plasmid, promoter (G) construct, promoter (T) construct. Transfections (n=12) were done using a Fugene-6 protocol according to the manufacture's specifications (Roche Applied Science, Mannheim, Germany). 48 hr after transfection, cells were washed with PBS twice and luciferase activities were measured with Dual-Luciferase Assay Kit (Promega, Madison, Wis., USA). Fold induction was derived relative to normalized reporter activity.

Computational Analysis of Transcription Factor Binding Sites in Rs1617640:

The possible transcript factor (TF) binding sites were examined in rs1617640 variants using the positional weighting matrices extracted from Matinspector. The TF binding sites for rs1617640 variants in a 10 bp flanking sequence are shown in Table 4.

ii. Results

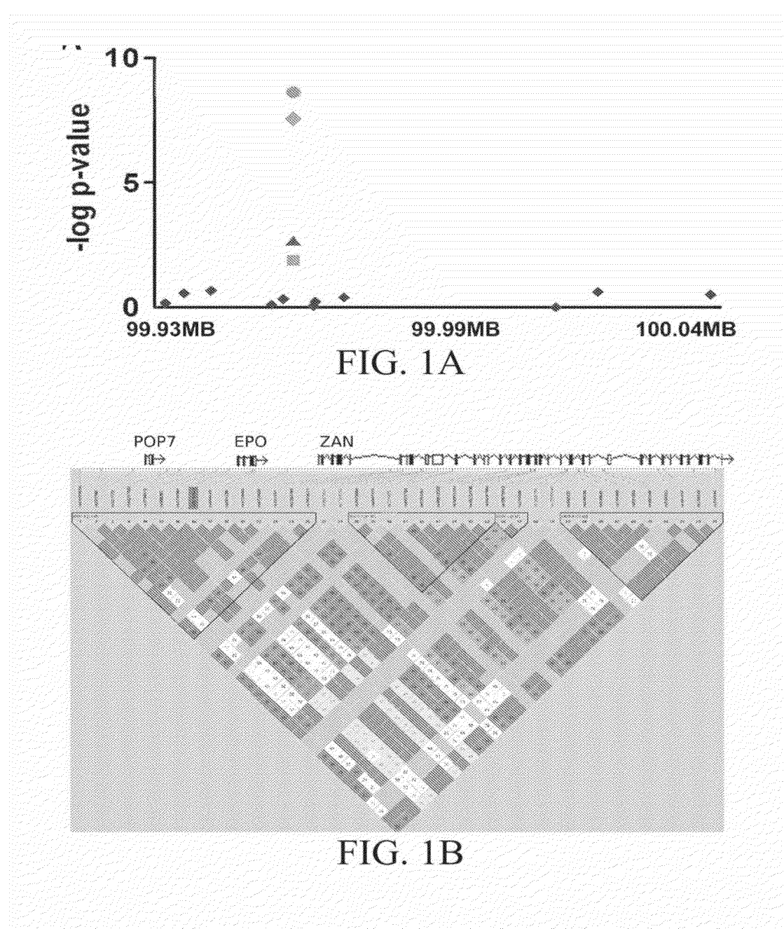

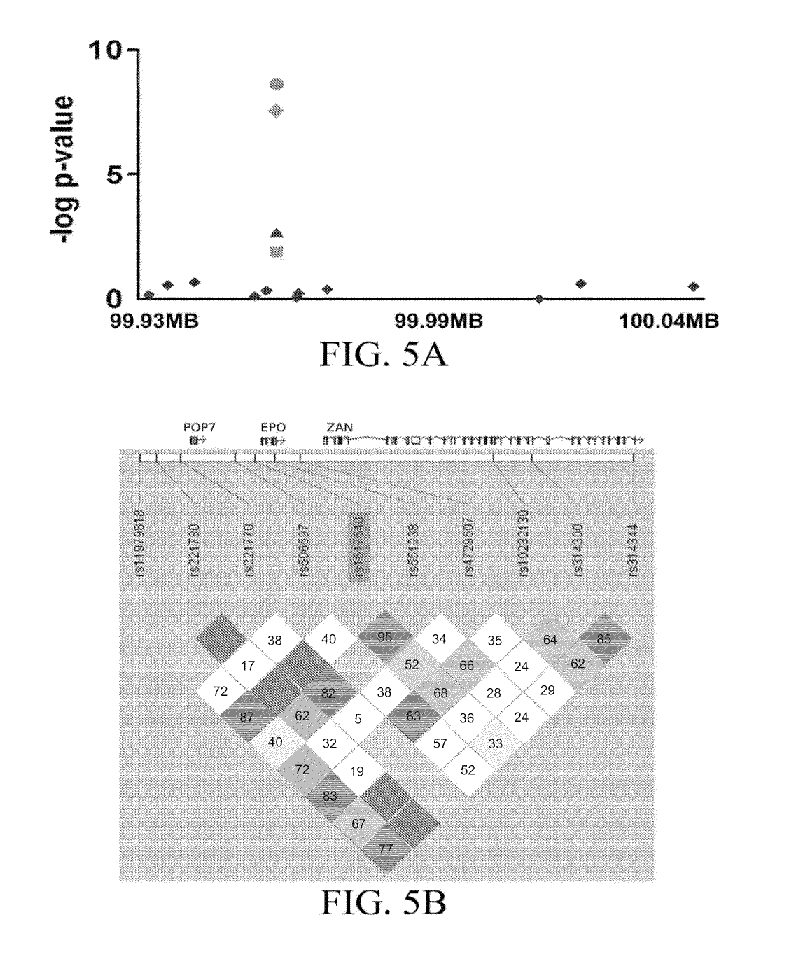

To identify a functional gene variant underlying the development of severe diabetic microvascular complications, 190 patients with diabetes and both documented ESRD and PDR in the GoKinD cohort were genotyped (Mueller et al., P. W. 2006). The GoKinD (Genetics of Kidneys in Diabetes) study is a collection of North American probands with type I diabetes, with and without diabetic nephropathy. (Mueller et al., P. W. 2006). Comparison was made with 179 diabetic control patients without any nephropathy or retinopathy in the same GoKinD cohort (Table 1) using a panel of 19 SNPs in the 11 candidate genes involved in angiogenesis. No significant association was found with SNPs in 10 of 11 genes (P>0.1, Table 3). However, rs1617640 located in the Epo gene promoter showed significant association (FIG. 1A, p=1.80×10⁻³ for an additive allele-dosage model). To further characterize these findings, second stage genotyping was performed with an additional 130 cases and 270 controls in the GoKinD cohort. This resulted in an increase in significance (p=4.07×10⁻⁷, ORₕₑₜ=1.11 [0.74, 1.68], ORₕom=2.52 [1.63, 3.91], T allele: 64.16% in cases versus 51.89% in controls). Nine additional tagging SNPs in Epo and adjacent genes (3 SNPs in Epo, 3 SNPs in POP7, and 3 SNPs in Zan) were then genotyped in 200 cases and 200 controls in the GoKinD cohort. In control samples, rs1617640 exhibited high linkage disequilibrium (LD) with its adjacent SNPs in Epo and the centromeric gene POP7, similar to the finding from the International HapMap CEU data (FIG. 1A). In contrast, rs1617640 in GoKinD cases is in low LD with its adjacent SNPs in Epo and POP7 and showed the strongest association signal (FIG. 5). To investigate whether rs1617640 is specifically associated with diabetic microvascular complications, rather than complications of type 1 diabetes (T1DM) per se, a type 2 diabetes (T2DM) cohort with ESRD and PDR (70 cases and 81 controls) from the Utah population were genotyped. A significant association was again demonstrated (P=0.013). When the GoKinD and Utah cohorts were combined for evaluation, rs1617640 showed a highly significant association (P=3.50×10⁻⁸). With an allele-dosage model, the estimated population attributable risk (PAR) for rs1617640 is 24.08%.

TABLE 1
Characteristics of PDR and ESRD Cases and Matched Controls
in GoKinD and Utah cohort.

| | | | | Average | Average | Average |
|---|---|---|---|---|---|---|
| | | Gender | Average | Duration | HbA 1 C | Hemoglobin |
| | n | (male/female) | Age (years) | (years) | (%) | (grams/deciliter) |
| GoKinD T1DMCase | 358 | 179/179 | 45 | 33 | 6.9 | NA |
| GoKinD T1DM | 490 | 192/298 | 39 | 25 | 7.4 | NA |
| Controls | | | | | | |
| Utah T2DM Cases | 78 | 41/37 | 67 | 24 | 6.8 | 12.0 |
| Utah T2DM Controls | 123 | 74/49 | 69 | 19 | 7.2 | 12.6 |

NA: data not available

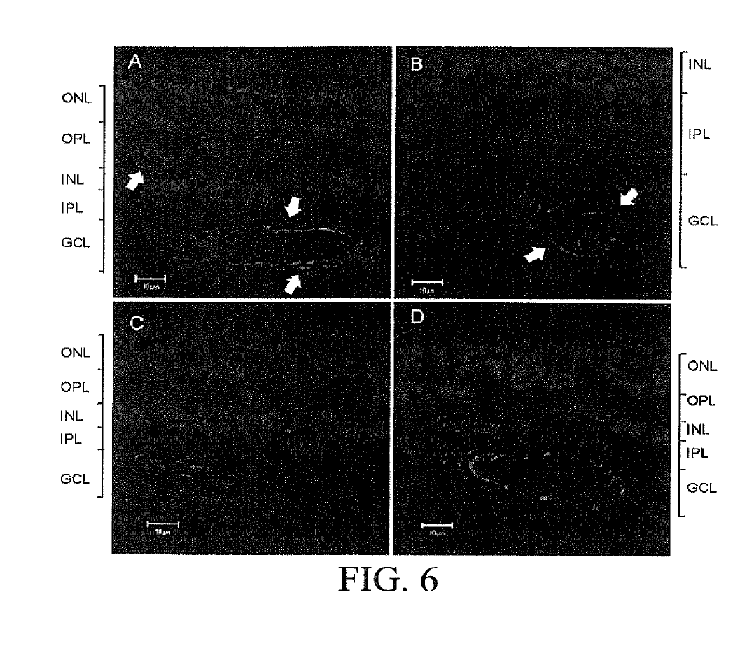

Epo encodes a potent angiogenic factor expressed in the retina and kidney (Watanabe et al., D. 2005). Epo concentrations were elevated in the vitreous of patients with PDR and more strongly associated with PDR than VEGF Inhibition of Epo in murine models prevents ischemia-induced retinal neovascularization (Watanabe et al., D. 2005). The SNP rs1617640 is located 1125 bp upstream to the Epo transcription start site. Using Matinspector to scan putative transcription factor binding sites within this region, we found that the risk allele T generates a core match with the EVII/MEL1 binding site or a matrix match with the AP1 binding site (Table 4). To demonstrate the functional significance of the associated T allele, expression studies were performed using real-time PCR with RNA derived from lymphocytes of cases from the Utah cohort (FIG. 2b). PDR and ESRD patients with the TT genotype had 4.8-fold higher Epo mRNA levels as compared to diabetic control patients with the GG genotype (FIG. 2b, p=0.033). In addition, Epo protein levels were 50-fold higher in vitreous samples of PDR and ESRD patients with a TT genotype than in diabetic control patients with a GG genotype (FIG. 2a, P=0.000046). Immunohistochemical evaluation demonstrated that Epo immuno labeling was increased in the adventitial layer of retina vasculature from a normal individual with TT genotype as compared to an eye from a normal individual with a GG genotype (FIG. 6).

TABLE 2
List of Primers used in SNP genotyping for candidate
genes involved in angiogenesis and Epo region

| | | | |
|---|---|---|---|
| | | FORWARD | |
| GENE | SNP | PRIMER | REVERSE PRIMER |

SNP's in candidate genes in angiogenesis

| | | | |
|---|---|---|---|
| ANGPT2 | rs2916702 | aagttggaaggaccacatgc | tagctagagcgagcacacact |
| | | (SEQ ID NO: 13) | (SEQ ID NO: 14) |
| ANGPT2 | rs2922881 | gatgagcaggatgcatgaga | cttgatgtctgtgggggact |
| | | (SEQ ID NO: 16) | (SEQ ID NO: 17) |
| Epo | rs1617640 | gtccattgtgcaggacacac | aactcctgggctcaaaggat |
| | | (SEQ ID NO: 19) | (SEQ ID NO: 20) |
| FGF1 | rs11747068 | gactcagtggcttggaggag | gccagatttggaagcatcat |
| | | (SEQ ID NO: 22) | (SEQ ID NO: 23) |
| FGF 1 | rs7708257 | ggctgtgcaatttaccacct | gtcacctgttccctgctgt |
| | | (SEQ ID NO: 25) | (SEQ ID NO: 26) |
| FGF2 | rs1264514 | aggagaatgctgcctctgaa | tctcgatctcctgacctcgt |
| | | (SEQ ID NO: 28) | (SEQ ID NO: 29) |
| FGF2 | rs17006255 | aacctccccacagaaaggat | cccctggattgtcactcatt |
| | | (SEQ ID NO: 31) | (SEQ ID NO: 32) |
| FLT1 | rs7993418 | tcctgaatccagaccctcac | gtccctctgggagtttcctc |
| | | (SEQ ID NO: 34) | (SEQ ID NO: 35 |
| FLTI | rs9513095 | gccaccgaagagtggaaata | ctacttgggaggctgaggtg |
| | | (SEQ ID NO: 37) | (SEQ ID NO: 38) |
| IGF1 | rs17796225 | gcggatgaaaaggaaactga | ttttggtctaccctgccaag |
| | | (SEQ ID NO: 40) | (SEQ ID NO: 41) |
| IGF1 | rs5742667 | ccctcccccatgaaaactat | ttcacagaagtgagcggttg |
| | | (SEQ ID NO: 43) | (SEQ ID NO: 44) |
| KDR | rs13109660 | aggcagaagtttccctggtt | aatccatgtgggagagttgc |
| | | (SEQ ID NO: 46) | (SEQ ID NO: 47) |
| KDR | rs1870378 | ccactacggcctcaatgagag | tgccaagtattgcatcgaga |
| | | (SEQ ID NO: 49) | (SEQ ID NO: 50) |
| Robo4 | rs12278566 | gtagaagggcacaagcaagc | aggaaacctgctttgctga |
| | | (SEQ ID NO: 52) | (SEQ ID NO: 53) |
| PEDF | rs4274475 | ctgagcgctaaaccagaacc | gcaccactgcacttccagtta |
| | | (SEQ ID NO: 55) | (SEQ ID NO: 56) |
| Slit2 | rs10516357 | tagattctggaattcctgtg | ggtaatttccaagatacaac |
| | | (SEQ ID NO: 58) | (SEQ ID NO: 59) |
| Slit2 | rs6828150 | gcctggattctggtgttgat | acgctgggtgtagggactta |
| | | (SEQ ID NO: 61) | (SEQ ID NO: 62) |
| TGFB1 | rs2241715 | tactcagcaaaccccaaagg | caatcctcttctccccaaca |
| | | (SEQ ID NO: 64) | (SEQ ID NO: 65) |
| TGFB1 | rs8179181 | tcctgccaactcacctctct | agggagacccagatggagat |
| | | (SEQ ID NO: 67) | (SEQ ID NO: 68) |

SNP's in Epo region

| POP7 | rs1197981 | acccaaatgatcctccttcc | acaccaggcactcaaattcc |
|---|---|---|---|
| | | (SEQ ID NO: 70) | (SEQ ID NO: 71) |
| POP7 | rs221780 | acctggctttctggatgcta | tcatcttgagggatccaagg |
| | | (SEQ ID NO: 73) | (SEQ ID NO: 74) |
| POP7 | rs221770 | gctgacctcagagcattcc | tcacataattgttgggagcg |
| | | (SEQ ID NO: 76) | (SEQ ID NO: 77) |
| Epo | rs506597 | taatccaggatgagccaagg | ctcccaaagtgctgggatta |
| | | (SEQ ID NO: 79) | (SEQ ID NO: 80) |
| Epo | rs551238 | cctgttttcgcacctaccat | tagcgaggatgtgcattgag |
| | | (SEQ ID NO: 82) | (SEQ ID NO: 83) |
| Epo | rs4729607 | tcccaaagtgctgggattac | gtcaggctggtctcgaactc |
| | | (SEQ ID NO: 85) | (SEQ ID NO: 86) |
| ZAN | rs10232130 | gggtgtcagagcaagactcc | agtggggacacacagaggac |
| | | (SEQ ID NO: 88) | (SEQ ID NO: 89) |
| ZAN | rs314300 | gcctttctgtgtagggtgga | gctgggcatcattagttcgt |
| | | (SEQ ID NO: 91) | (SEQ ID NO: 92) |
| ZAN | rs314344 | aggagttcaaggctgcagaa | tctctccttcccaggactca |
| | | (SEQ ID NO: 94) | (SEQ ID NO: 95) |
| | | GENE | SNAPSHOT PRIMER |

SNP's in candidate genes in angiogenesis

| | |
|---|---|
| ANGPT2 | ggattactttgcagtcccagcaacatggtgggctggactc |
| | (SEQ ID NO: 15) |
| ANGPT2 | gtaaatagttgctatactgtattgtttt |
| | (SEQ ID NO: 18) |
| Epo | ttatggcttctggaaaccctgagccaga |
| | (SEQ ID NO: 21) |
| FGF1 | ttaaccacttcctagctctgttcacatcaaatcg |
| | (SEQ ID NO: 24) |
| FGF 1 | gacacaggctacttgttccgaagttttaagctgtagatgttcccaa |
| | (SEQ ID NO: 27 |
| FGF2 | aatatctatctagtggaaacacaaaatgcttata |
| | (SEQ ID NO:30) |
| FGF2 | atttettetgaatgaagaccntggatttctaaaageaccaaaacaaggett |
| | (SEQ ID NO: 33) |
| FLT1 | ttttgattctttccaggctcatgaacttgaaagcatttac |
| | (SEQ ID NO: 36) |
| FLTI | aaattgatgaaaatggtaaactgtggcc |
| | (SEQ ID NO: 39) |
| IGF1 | atgcagcagcctctaagtca |
| | (SEQ ID NO: 42) |
| IGF1 | gtatgatttctcctgccaatatcataagctgagaatattt |
| | (SEQ ID NO: 45) |
| KDR | ttagcttctttcttcctcaaggactttc |
| | (SEQ ID NO: 48) |
| KDR | taaatagagaattacttccctaggaccaccaga |
| | (SEQ ID NO: 51) |
| Robo4 | acatagcaattcatcaaacaacagcgtgcttccaagtcacc |
| | (SEQ ID NO: 54) |
| PEDF | ctcaaagacgggatgcttgtcgtcgagtctcata |
| | (SEQ ID NO: 57) |
| Slit2 | ataggcttgagtgggagcttttgttctgtgtcttaaaattattgatattgtc |
| | (SEQ ID NO: 60) |
| Slit2 | tcgttgtatgaattttagcattacagataccacacctattagatatgggcaggacta |
| | (SEQ ID NO: 63) |
| TGFB1 | cagagagggagaggcgggagatgtcagagacggagacgaggcaac |
| | (SEQ ID NO: 66) |
| TGFB1 | cgcacgcagcagttcttctccgtggagctgcaggcaggagagacgc |
| | (SEQ ID NO: 69) |

SNP's in Epo region

| | |
|---|---|
| POP7 | ctttggaagttactccagaatgtataccttactt |
| | (SEQ ID NO: 72) |
| POP7 | gatgtgagccactgtgcccgacccatatcctgttccaata |
| | (SEQ ID NO: 75) |
| POP7 | ccctgcaactgctacaattaacaggatatgggagagaggaggcacatcatgtg |
| | (SEQ ID NO: 78) |
| Epo | gagggtctcagttctgaggcaataggaagccgtg |
| | (SEQ ID NO: 81) |
| Epo | gggcagggttggcagctgccttactgcggtgaggccttgaatggag |
| | (SEQ ID NO: 84) |
| Epo | agacactggaatctaagctgaaggctaa |
| | (SEQ ID NO: 87) |
| ZAN | gttgttgttgtagttccctagaggagggagtcattaaaacaggcaa |
| | (SEQ ID NO: 90) |
| ZAN | caggtctgcggcatgtgtgggaacttca |
| | (SEQ ID NO: 93) |
| ZAN | ctctctggtcacggccagtagtacgaccaccacaggcaccagcagtcccagtaggacg |
| | (SEQ ID NO: 96) |

TABLE 3
Association results for SNP's in 11 candidate genes involved in
angiogenesis in ESRD and PDR cases and controls in the GoKinD cohort.

| | | | | | MAF |
|---|---|---|---|---|---|
| Gene | SNP | p-value | Function | MAF Case | Control |
| ANGPT2 | rs2916702 | 0.339 | ntron 9 | 0.43 | 0.37 |
| ANGPT2 | rs2922881 | 0.182 | intron 9 | 0.20 | 0.28 |
| Epo | rs1617640 | 1.80E−03 | Promoter | 0.31 | 0.44 |
| FGF1 | rs11747068 | 0.721 | intron 1 | 0.20 | 0.17 |
| FGF1 | rs7708257 | 0.646 | intron 1 | 0.25 | 0.30 |
| FGF2 | rs12640514 | 0.299 | intron 1 | 0.38 | 0.29 |
| FGF2 | rs17006255 | 0.682 | intron 2 | 0.18 | 0.18 |
| FLT1 | rs7993418 | 0.135 | Y1213Y | 0.29 | 0.23 |
| FLT1 | rs9513095 | 0.631 | intron 12 | 0.30 | 0.32 |
| IGF1 | rs17796225 | 0.675 | intron 2 | 0.24 | 0.26 |
| IGF1 | rs5742667 | 0.516 | intron 2 | 0.26 | 0.32 |
| KDR | rs13109660 | 0.249 | intron 13 | 0.36 | 0.33 |
| KDR | rs1870378 | 0.119 | intron 15 | 0.30 | 0.20 |
| ROBO4 | rs12278566 | 0.609 | intron 7 | 0.17 | 0.13 |
| PEDF | rs4274475 | 0.887 | intron 4 | 0.26 | 0.29 |
| SLIT2 | rs10516357 | 0.688 | intron 26 | 0.34 | 0.37 |
| SLIT2 | rs6828150 | 0.514 | intron 4 | 0.31 | 0.28 |
| TGFB1 | rs2241715 | 0.488 | intron 1 | 0.15 | 0.25 |
| TGFB1 | rs8179181 | 0.442 | intron 5 | 0.24 | 0.19 |

TABLE 4
Matrices for rs1617640 variants binding sites in the Epo-1135/−1114 promoter region

| | | Position | Core | Matrix | |
|---|---|---|---|---|---|
| rs1617640 | Matrix identifier | (strand) | match | match | Sequence |
| G | V$BRAC/BRACH.01 | 1-21 (−) | 0.750 | 0.660 | ctgagccagAGGAgtgagatt |
| | | | | | (SEQ ID NO: 97) |
| T | V$SEV11/MEL1.02 | 2-18 (−) | 1.000 | 0.997 | tgagccaGATGagtgag |
| | | | | | (SEQ ID NO: 98) |
| T | V$AP1F/AP1.02 | 9-19 (−) | 1.000 | 0.909 | ga [symbol] GAGTgag |
| | | | | | (SEQ ID NO: 99 |

Notes:
Italics indicates the matrix match to binding site. Uppercase represents the core match. The
variants of rs167640 are bolded and underlined.

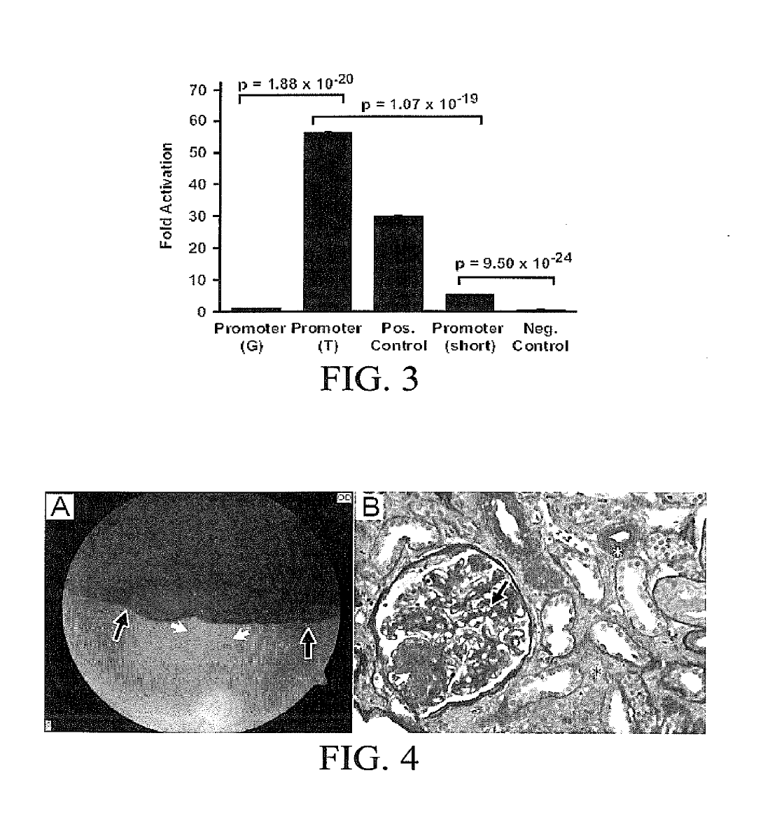

To further demonstrate the effect of the T allele on transcription regulation, an in vitro promoter reporter assay was performed in a cell transfection experiment. T allele markedly enhanced expression of Luciferase reporter expression by 55-fold compared to that of the G allele (FIG. 3, P=1.88×10″²⁰). Consistent with the hypothesis that increased expression of Epo plays a role in the pathogenesis of diabetic microvascular complications, it was demonstrated that the expression of Epo was significantly increased in the kidney of db/db mice (FIG. 2C, P=0.0022) and in the mouse ischemic retina (FIG. 2D, P=1.1×10⁻′).

Epo stimulates erythropoiesis and production of red blood cells. It is widely used to treat anemia resulting from renal failure or cancer chemotherapy. In the United States, erythropoietin represents the largest single drug expense for the Center for Medicare & Medicaid Services, approximately one billion dollars per year. Total sales of Epo agents by Amgen and Johnson & Johnson are believed by analysts to have exceeded $8.6 billion in 2004 (Steensma, D. P. 2005).

Recent evidence indicates that patients with anemia due to chronic renal disease (many of whom have diabetes) who receive frequent dosing of Epo to maintain higher hemoglobin (13.5 g per deciliter) have a higher rate of both progression to ESRD and cardiovascular complications as compared with those patients maintaining a lower hemoglobin level (11.3 g per deciliter)(Singh et al., A. K. 2006). A similar effect of Epo on kidney function had also been suggested by earlier studies (Garcia, D. L. 1988). The disclosed genotype risk results indicate that care is warranted when maintaining higher hemoglobin concentration using exogenous Epo treatment in diabetic patients as it might accelerate progression to end-stage renal disease and PDR.

In the eye, Epo concentrations are markedly elevated and are more strongly associated with presence of PDR than is VEGF (Mueller et al., P. W. 2006). In the murine ischemic retinopathy model (Smith et al., L. E. 1994), Epo inhibition reduces development of retinal neovascularization (Mueller et al., P. W. 2006). Together with the disclosed results, these findings indicate that localized inhibition of Epo activity in the eye can have therapeutic potential for the treatment or prevention of PDR.

Severe diabetic microvascular complications including both PDR and ESRD are also strong predictors of cardiovascular disease and mortality and are associated with less than a 10% 10-year survival rate (Adler et al., A. I. 2003). Thus, identification of patients with a strong genetic risk and implementation of preventative measures are of considerable importance. The disclosed findings strongly support a key role for Epo in genetic susceptibility to PDR and ESRD and identify a new pathogenetic mechanism for advanced diabetic microvascular complications.

### G. REFERENCES

Abramson, J. H. and Gahlinger, P. M. PEPI, ver. 4.0: Computer Programs for Epidemiologists. (Sagebrush Press, Salt Lake City, 2001).
Adler A. I. et al., Kidney Int 63, 225 (January, 2003).
Aiello L. P. et al., N Engl J Med 331, 1480 (Dec. 1, 1994).
Aiello, L. P. N Engl J Med 353, 839 (Aug. 25, 2005).
Barrett, J. C., et al. Bioinformatics 21, 263 (Jan. 15, 2005).
Bowden D. W. et al., Kidney Int 66, 1517 (October, 2004).
Chistiakov D. A. et al., Diabetes Res Clin Pract 66, 79 (October, 2004).
Gabriel S. B. et al., Science 296, 2225 (Jun. 21, 2002).
Garcia D. L., et al. Proc Nall Acad Sci USA 85, 6142 (August, 1988). Girach, A. D., et al. Int J Clin Pract 60, 1471 (November, 2006). Kramer, H. J., et al. JAMA 289, 3273 (Jun. 25, 2003).
Levin, M. L. Acta Unio hit Contra Cancrum 9, 531 (1953). Moczulski, D. K., et al. Diabetes 47, 1164 (July, 1998).
Mueller P. W. et al., J Am Soc Nephrol 17, 1782 (July, 2006).
Mueller P. W. et al., J Am Soc Nephroi 17, 1782 (July, 2006).
Parving, H.-H., et al. in The Kidney B. M. Brenner, Ed. (Elsevier, Philadelphia, 2004) pp. 1777-1818.
Reichard, P. J Diabetes Complications 9, 25 (January-March, 1995). Rich, S. S. J Am Soc Nephrol 17, 353 (February, 2006).
Sharma, K., et al. Am J Physiol Renal Physiol 284, F1138 (June, 2003). Singh A. K. et al., N Engl J Med 355, 2085 (Nov. 16, 2006). Smith L. E. et al., Invest Ophthalmol V is Sci 35, 101 (January, 1994). Smith L. E. et al., Invest Ophthalmol V is Sci 35, 101 (January, 1994). Steensma, D. P., et al. J Clin Oncol 23, 5865 (Sep. 1, 2005). Watanabe D. et al., N Engl J Med 353, 782 (Aug. 25, 2005). Yang Z. et al., Science 314, 992 (Nov. 10, 2006).

1. A method of identifying a subject at risk for developing a microvascular complication associated with diabetes mellitus, the method comprising:
obtaining a nucleic acid sample from a subject;
genotyping the nucleic acid sample for the presence of at least one single nucleotide polymorphism at a position located approximately 1125 nucleotide base pairs upstream from the erythropoietin gene (Epo) transcription start site, wherein the at least one single nucleotide polymorphism is a thymidine at 1125 base pairs upstream from the Epo transcription start site; and
identifying the subject with the at least one single nucleotide polymorphism at a position located approximately 1125 nucleotide base pairs upstream from the Epa transcription start site as being at risk for developing a microvascular complication associated with diabetes mellitus.

2. (canceled)

3. The method of claim 1, wherein the at least one single nucleotide polymorphism at a position located approximately 1125 nucleotide base pairs upstream from the erythropoietin gene (Epa) transcription start site is at position 100155234 of human chromosome 7.

4. The method of claim 1, wherein the at least one single nucleotide polymorphism is detected by a process comprising
(a) providing a probe that hybridizes under stringent conditions to an oligonucleotide consisting of SEQ ID NO: 1 but does not hybridizes under stringent conditions to an oligonucleotide consisting of SEQ ID NO:2, and
(b) detecting hybridization of said probe to the nucleic acid sample.

5. The method of claim 1, wherein the subject has been diagnosed with type 1 or type 2 diabetes mellitus.

6. The method of claim 1, wherein the thymidine is detected by gene sequencing.

7. The method of claim 1, wherein the microvascular complication is nephropathy.

8. The method of claim 1, wherein the microvascular complication is retinopathy.

9. The method of claim 3, wherein a treatment protocol is chosen for the subject based on the detection of thymidine at position 100155234 of human chromosome 7.

10. The method of claim 1, further comprising administering to the subject a therapeutically effective amount of an erythropoietin inhibitor.

11. A method of identifying a subject at risk for developing a microvascular complication associated with diabetes mellitus, the method comprising:
obtaining a sample of nucleic acid from a subject diagnosed with diabetes mellitus;
detecting in the nucleic acid sample a single nucleotide polymorphism by providing a probe that hybridizes under stringent conditions to an oligonucleotide consisting of SEQ ID NO: 1 but does not hybridizes under stringent conditions to an oligonucleotide consisting of SEQ ID NO:2, wherein the at least one single nucleotide polymorphism is a thymidine at 1125 base pairs upstream from the Epo transcription start site, and detecting hybridization of said probe to the nucleic acid sample; and
wherein hybridization of the probe to the nucleic acid sample detects a single nucleotide polymorphism which identifies a subject at risk for developing a microvascular complication associated with diabetes mellitus.

12. The method of claim 11, wherein the probe comprises SEQ ID NO: 98.

13. The method of claim 11, wherein the single nucleotide polymorphism is a thymidine located at position 100155234 of human chromosome 7.

14. The method of claim 11, wherein the nucleic acid sample from the subject diagnosed with diabetes comprises extracted genomic DNA that is PCR-amplified with the forward primer SEQ ID NO: 9 and the reverse primer SEQ ID NO: 10.

15. The method of claim 11, wherein the nucleic acid sample from the subject diagnosed with diabetes comprises extracted genomic DNA that is KR-amplified with the forward primer SEQ ID NO: 19 and the reverse primer SEQ ID NO: 20.

16. The method of claim 11, wherein position 100155234 of human chromosome 7 is 1125 nucleotide base pairs upstream from the erythropoietin gene (Epo) transcription start site.

17. The method of claim 11, wherein the microvascular complication is nephropathy.

18. The method of claim 11, wherein the microvascular complication is retinopathy.

19. The method of claim 18, wherein the microvascular complication is proliferative diabetic retinopathy (PDR).

20. The method of claim 11, wherein the identified subject is monitored for levels of erythropoietin.

21. The method of claim 13, wherein a treatment protocol is chosen for the subject based on the detection of thymidine at position 100155234 of human chromosome 7.

22. (canceled)
